(19) United States
(12) Patent Application Publication
(10) Pub. No.: US 2022/0205982 A1
(43) Pub. Date: Jun. 30, 2022

(54) METHODS FOR DETERMINING PERSONALIZED FULL DOSE OF MELPHALAN IN REDUCED INTENSITY REGIMEN PRIOR TO HEMATOPOIETIC CELL TRANSPLANTATION

(71) Applicant: CHILDREN'S HOSPITAL MEDICAL CENTER, Cincinnati, OH (US)

(72) Inventors: Parinda MEHTA, Cincinnati, OH (US); Kenneth D.R. SETCHELL, Cincinnati, OH (US); Alexander A. VINKS, Cincinnati, OH (US)

(73) Assignee: CHILDREN'S HOSPITAL MEDICAL CENTER, Cincinnati, OH (US)

(21) Appl. No.: 17/611,398

(22) PCT Filed: May 15, 2020
(86) PCT No.: PCT/US2020/033178

§ 371 Date: Nov. 15, 2021

Related U.S. Application Data
(60) Provisional application No. 62/848,928, filed on May 16, 2019.

(51) Int. Cl. G01N 33/50; A61K 31/198; A61K 39/395; A61K 31/7076; A61K 35/28; A61P 37/06

(52) U.S. Cl. CPC G01N 33/5088; A61K 31/198; A61K 39/3955; G01N 2800/52; A61K 35/28; A61P 37/06; G01N 2800/22; A61K 31/7076

(57) ABSTRACT

A method for determining a personalized full dose of a melphalan compound (e.g., melphalan) in a reduced intensity conditioning regimen (RIC) prior to hematopoietic cell transplantation for a subject based on pharmacokinetic features of the melphalan compound administered to the subject at a test dose.

### CROSS-REFERENCE TO RELATED APPLICATIONS

The present application claims the benefit under 35 USC § 119(e) to U.S. Provisional Application No. 62/848,928, filed on May 16, 2019, which is hereby incorporated by reference in its entirety.

### BACKGROUND OF THE INVENTION

Hematopoietic cell transplantation (HCT) such as hematopoietic stem cell transplantation (HSCT) continues to be the only curative therapy for patients with many hematological diseases. Melphalan is an alkylating agent that has demonstrated activity against a number of malignant diseases and at high doses. It is an important component of many HSCT preparative regimens. Shaw et al., Bone Marrow Transplant; 16: 401-5 (1996). Common toxicities observed with melphalan use in this setting include: gastrointestinal tract toxicity including severe mucositis with vomiting, diarrhea, gastrointestinal bleeding, veno-occlusive disease, and renal insufficiency including renal failure, which at times can be life threatening, affecting overall transplant outcome. Samuels et al., J Clin Oncol.; 13:1786-1799 (1995).

Thus, it is of importance to develop methods for assessing suitable doses of melphalan for use in conditioning a patient prior to HSCT so as to achieve the desired therapeutic effects while reducing or eliminating toxicities caused by melphalan.

### SUMMARY OF THE INVENTION

The present disclosure is based, at least in part, on the development of a method for determining a suitable personalized dose (a.k.a., precision dosing) of a melphalan compound for a specific patient to minimize toxicity caused by the compound and achieving the intended effect of destroying bone marrow cells of the subject to facilitate the following hematopoietic cell transplantation.

Accordingly, one aspect of the present disclosure provides a method for determining a personalized full dose of a melphalan compound for a subject in a reduced intensity conditioning regimen (RIC), optionally prior to hematopoietic cell transplantation. Such a method may comprise: (i) administering to the subject in need thereof (e.g., a subject in need of hematopoietic cell transplantation) a test dose of the melphalan compound, (ii) collecting blood samples before administration of the test dose of the melphalan compound and at multiple time points after administration of the test dose of the melphalan compound; (iii) measuring the levels of the melphalan compound or a metabolite thereof in the blood samples; (iv) calculating pharmacokinetic features of the melphalan compound based on the levels of the melphalan compound or the metabolite thereof measured in step (iii); and (v) determining a personalized full dose of the melphalan compound in the RIC for the subject based on the pharmacokinetic features calculated in step (iv). In some instances, the test dose of the melphalan compound can be about 10% to about 30% (e.g., about 10% or about 20%) of a standard full dose of the melphalan compound for use in a RIC.

In some embodiments, the pharmacokinetic features of the melphalan compound comprise area under the curve (AUC). In some examples, the AUC can be calculated by the trapezoidal method. In other embodiments, the pharmacokinetic features of the melphalan compound comprise median clearance (CL). For example, the median clearance can be median body weight normalized clearance (CL_{STD}). In some examples, the pharmacokinetic features of the melphalan compound comprise both AUC and CL (e.g., CL_{STD}).

The subject may be a human patient having a non-malignant disorder, for example, a hematologic disease. Examples include, but are not limited to, an immune deficiency disorder (e.g., a disorder associated with immune dysregulation), a hemoglobinopathy (e.g., sickle cell disease), bone marrow failure (e.g., congenital or acquired), anemia (e.g., aplastic anemia), or a genetic metabolic disorder. In some instances, the subject may be a human patient having hemophagocytic lymphohistiocytosis, combined immune deficiency (e.g., severe combined immune deficiency), IPEX Syndrome (Immune dysregulation, polyendocrinopathy, enteropathy, X-linked Syndrome), or erythropoietic protoporphyria.

The subject may be a human child (e.g., a child younger than 5 years old). In some instances, the subject can be a human infant. Alternatively or in addition, the subject may have a body weight lower than 10 kg. In other embodiments, the subject can be a human adult. For child subjects, the test dose may be about 30% of the standard full dose of the melphalan compound, for example, melphalan.

In some instances, any of the subject disclosed herein (e.g., a human patient) may have an organ dysfunction. For example, the subject may have liver dysfunction, kidney dysfunction, severe colitis, respiratory failure, cardiac dysfunction, or a combination thereof.

In any of the methods disclosed herein, the blood samples can be collected before administration of the melphalan compound and at multiple time points, e.g., at about 5 minutes, about 15 minutes, about 30 minutes, about 45 minutes, about 60 minutes, about 2 hours, about 2.5 hours, about 4 hours, and about 6 hours. Alternatively, the blood samples can be collected at about 0.08 hour, 0.5±0.1 hour, 1.5±0.3 hours, and 4.0 hours after the administration of melphalan. In another example, the blood samples can be collected between 0.08-0.19 hour, 0.33-0.90 hour, 1.3-2.7 hours, and 3.6-4.0 hours after the administration of the melphalan compound.

In some embodiments, the RIC further comprises alemtuzumab and fludarabine and at least a portion of the multiple blood samples in step (ii) can be collected after administration of the alemtuzumab and/or fludarabine.

Alternatively or in addition, the levels of the melphalan compound or the metabolite thereof is determined by LC-MS/MS or paper spray (PS)-MS/MS.

In any of the methods disclosed herein, the personalized full dose of the melphalan compound determined in step (v) can be based further on one or more characteristics of the subject (e.g., a human patient such as a human child). Such characteristics may comprise one or more of the following: age, weight, disease condition, organ function, blood cell count, bone marrow cellularity, infectious status, congenital anomaly, and clinical status. For example, organ function may comprise liver function, kidney function, digestive tract function, lung function, cardiac function, or a combination thereof. In some embodiments, the personalized full dose determined in step (v) can be predicted to result in a target AUC of about 3.5-6.5 h*μg/mL in the subject, who has normal organ function.

Any of the methods disclosed herein may further comprise (vi) subjecting the subject to a RIC comprising melphalan, wherein the subject is administered with the melphalan at the personalized full dose determined in step (v). In some examples, the RIC may further comprise alemtuzumab and fludarabine. Optionally, the method may further comprise subjecting the subject to hematopoietic cell transplantation after step (vi).

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a diagram showing enrollment details of the patients. Of the 26 patients enrolled, 23 patients received both test dose and full dose of melphalan, 2 patients received full dose of melphalan only, and one patient received test dose of melphalan only.

FIG. 2 is a diagram showing individual observed PK profiles following the test dose of melphalan, the full dose of melphalan, and the test dose predicted profile. The data presented plots the melphalan concentration in plasma (mg/L) versus time.

FIGS. 3A and 3B include diagrams showing a comparison between the melphalan test dose PK parameters versus the full dose PK parameters. 3A: a diagram showing test dose-predicted versus observed area under curve (AUC). The blue shaded area is the proposed target AUC range in this population (3.5-6.5 mg·h/L). Open circles represent patients given standard full dose (4.7 mg/kg for <10 kg, 140 mg/m² for ≥10 kg) and closed circles represent patients given PK adjusted dose (n=7). 3B: diagrams showing comparisons between test dose PK parameters versus full dose PK parameters as indicated.

FIG. 4 is a bar graph detailing the prediction performance of the test dose PK parameters.

FIGS. 5A-5C include diagrams showing the prediction error difference by bodyweight. 5A: diagrams showing comparison of test dose PK parameters versus full dose PK parameters. 5B: diagrams showing prediction errors comparing the melphalan full dose AUC versus the melphalan test dose AUC, the melphalan full dose CL_{STD} versus the melphalan test dose CL_{STD}, and the melphalan full dose V_{C} versus the melphalan test dose V_{C} in subjects having different body weights as indicated. 5C: diagrams showing prediction error percentages in subjects having body weight lower than 10 kg or equal to or greater than 10 kg.

FIGS. 6A-6C include diagrams showing the prediction error difference by age. 6A: cutoff age of 1 year. 6B: cutoff age of 2 years. 6C: cutoff age of 5 years.

FIGS. 7A and 7B include graphs showing the AUC parameters in patients receiving the PK-guided dose adjustment of melphalan. The correlation of in receiving PK-guided dose adjustment of melphalan was good (R²=0.78). 7A: test dose predicted AUC versus observed AUC. 7B: a chart showing prevention of overexposure by PK-guided dose.

FIG. 8 is a schematic illustration of paper spray for MS analysis.

FIG. 9 includes diagrams showing Paper Spray Ionization CID mass spectra and chemical structures of melphalan (upper panel) and the internal standard (B) [²H₈]-melphalan (lower panel), showing key fragmentations and highlighting the selected quantifier and qualifier ion transitions that were monitored.

FIGS. 10A-10B include diagrams showing representative PS-MS/MS data for a patient sample. 10A: extracted ion chronograms for melphalan (upper panel) and [²H₈]-melphalan (lower panel). Calibration curves over a large dynamic range of melphalan concentrations. 10B: the SRM spectra for the quantifier ion (m/z 305.1→246.2 for melphalan and m/z 313.1→254.2 for [²H₈]-melphalan) and qualifier ion (m/z 305.1→194.2 for melphalan and m/z 313.1→200.2 for [²H₈]-melphalan) and the ion ratio.

FIG. 11 includes calibration curves for whole blood melphalan over a large dynamic range of melphalan concentrations measured by PS-MS/MS. Upper panel: a whole range of the calibration curve. Bottom panel: expanded version of the calibration curve portion as indicated in the upper panel.

FIGS. 12A-12D include diagrams showing comparison among an HPLC-MS/MS assay, a paper spray MS/MS (PS-MS/MS) assay, and a liquid chromatography tandem mass spectrometry (LC-MS/MS) assay as disclosed herein. 12A: a comparison of PS-MS/MS for blood vs. HPLC-MS/MS for plasma. 12B: a comparison of PS-MS/MS for plasma vs. HPLC-MS/MS for plasma. 12C: a comparison of PS-MS/MS for blood vs. LC-MS/MS for plasma with sample size n=192. 12D: a comparison of PS-MS/MS for blood vs. LC-MS/MS for plasma with sample size n=208.

FIG. 13 includes diagrams showing melphalan PK behavior for 5 patients (panels 1-5, respectively) after intravenous administration of standard full dose by PS-MS/MS (blood) and LC-MS/MS (plasma) methods. The black circles in this figure are LC-ESI/MS/MS in blood plasma. The red circles are PS-MS/MS in whole blood.

FIG. 14 is a diagram showing the comparison between calculated AUCs by PS-MS/MS (blood) and calculated AUCs by LC-MS/MS (plasma).

### DETAILED DESCRIPTION OF THE INVENTION

High dose melphalan (HDM) is an important component of hematopoietic cell transplant (HCT) preparative regimens for both autologous and allogenic transplants (e.g., for multiple myeloma, solid tumors and hematological malignancies). In this setting, melphalan is usually administered at doses ranging from 140 to 200 mg/m². Bayraktar et al., Biol Blood Marrow Transplant (2013) 19:344-356. At these doses, melphalan is known to be associated with significant non-hematological toxicity including moderate to severe mucositis, gastrointestinal bleeding, veno-occlusive disease (VOD) of the liver, significant burn like skin rashes, pneumonitis and renal insufficiency. Samuels et al., J Clin Oncol. (1995) 13:1786-1799. Pharmacokinetic (PK) studies in adults with multiple myeloma undergoing autologous HCT have correlated toxicity with increased systemic exposure to melphalan. Nath et al., Br J Clin Pharmacol. (2016) 82:149-159.

There are no melphalan PK data in children or adults undergoing allogeneic HCT for non-malignant disorders, using reduced intensity conditioning (RIC), where HDM is commonly employed along with fludarabine and alemtuzumab. In this setting, the primary role of high dose melphalan is to create space in the bone marrow to facilitate engraftment of donor cells and the typical dose used (known as a standard full dose) is 140 mg/m² for human subjects having a body weight ≥10 kg or 4.7 mg/kg for human subjects having a body weight <10 kg. In adults undergoing autologous HCT for multiple myeloma, significant inter-patient variability (up to 5 fold) in melphalan exposure has been reported. Nath et al., Br J Clin Pharmacol. (2016) 82:149-159. Therefore, the current fixed dosing approach based on body surface area or weight may not be optimal for everyone. Moreover, in RIC HCT for non-malignant disorders, excess systemic exposure is not desirable, unlike in HCT for malignant disorders, where an improved disease control can somewhat justify additional toxicity related to increased systemic exposure.

Additionally, renal clearance is an important route of melphalan elimination and in adults undergoing autologous HCT with renal impairment for multiple myeloma, high dose melphalan was associated with increased short-term toxicity. Sweiss et al., Bone Marrow Transplant. (2016) 51:1337-1341. Individualized PK directed melphalan dosing is therefore necessary to limit inter-patient variability in drug exposure, and to achieve a desired level of melphalan exposure that would ensure successful engraftment while minimizing toxicity, particularly in children with organ impairment.

To address the melphalan dosing problem in association with RIC-HCT for treating non-malignant disorders, the present disclosure provides melphalan dose optimization approaches based on pharmacokinetic (PK) studies of a test dose given to a candidate patient prior to start of conditioning. PK features of both test dose and standard full dose melphalan in patients undergoing RIC-HCT for non-malignant disorders using a uniform alemtuzumab, fludarabine and melphalan regimen were studies. Results obtained from the current studies show that test dose PK can reliably predict standard full dose PK and would allow dose adjustment of standard full dose melphalan in patients, particularly in children, undergoing HCT with both normal and impaired organ function. See Example below. Given the common use of melphalan in conditioning regimens for HCT (e.g., HSCT), especially RIC regimens for allogeneic HCT for malignant and non-malignant diseases, predicting a suitable full dose of a nitrogen mustard alkylating agent such as a melphalan compound in an RIC regimen based on individualized PK and/or patient characteristics, would limit inter-patient variability, minimize toxicity, and improve clinical outcomes. As used herein, a personalized full dose of a melphalan compound for a subject refers to a suitable dose of the melphalan compound specific to the subject that minimizes development of toxicity caused by the melphalan compound and is sufficient to achieve the conditioning effects as part of a RIC regimen.

Also provided herein is an improved method of measuring melphalan in whole blood in clinical settings. Typically, melphalan is measured in plasma using a liquid chromatography tandem mass spectrometry (LC-MS/MS) assay. Mirkou et al., J Chromatogr B Analyt Technol Biomed Life Sci., 877:3089-3096 (2009). However, this assay requires complex sample preparation and has a long chromatographic run time. Paper spray (PS) is an ionization method that allows rapid quantitative analysis of drugs by mass spectrometry (PS-MS/MS) directly from whole blood without the need for prior sample preparation or separation. Liu et al., Anal Chem., 82:2463-2471 (2010). The entire analysis time is only a few minutes, thereby permitting real-time analysis and rapid data reporting. In addition, compared to the conventional LC-MS/MS methods, this improved method only requires a small volume of blood (a drop of blood), which is a significant advantage for PK studies in young children.

### I. Predicting a Personalized Full Melphalan Dose for Reduced Intensity Conditioning Regimen Based on Test Dose Pharmacokinetic Characteristics

In some aspects, the present disclosure provides a method for predicting a personalized full dose of a nitrogen mustard alkylating agent (e.g., a melphalan compound) as part of a reduced intensity conditioning (RIC) regimen for individual subjects (e.g., human patients such as children or adults). In some instances, the subjects may be in need of hematopoietic cell transplantation (HCT) such as hematopoietic stem cell transplantation. The RIC regimen can be performed prior to the transplantation for conditioning the subject for the HCT. In some instances, the HCT is for treatment of a non-malignant disorder. In other instances, the RIC regimen may not be performed in association with HCT. For example, the RIC regimen may be performed to a subject in need of gene therapy.

In this method, a test dose of melphalan can be given to a subject (e.g., who needs HCT treatment, e.g., for a non-malignant disorder) via a routine administration route. Blood samples can be collected from the subject before the administration of the test dose and at one or more time points after the administration. The level of melphalan or a metabolite thereof in the blood samples can be measured and pharmacokinetic (PK) features of the melphalan compound (e.g., melphalan) can be calculated based on the levels of the melphalan compound or its metabolite in the blood samples. A personalized full dose of melphalan for that specific subject can then be determined based on the PK features, and optionally also take into consideration the subject's clinical characteristics (e.g., those described herein).

Nitrogen mustard alkylating agents, derived from mustard gas, are a group of compounds capable of alkylating DNA and form inter-strand cross-links in DNAs. Such compounds are commonly used in cancer therapy. Nitrogen mustard alkylating agents typically contain the core structure of

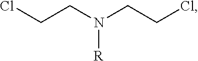

in which R is optionally substituted carbocyclyl, optionally substituted heterocyclyl, optionally substituted aryl, or optionally substituted heteroaryl. In some instances, R is optionally substituted carbocyclyl, optionally substituted aryl (e.g., substituted phenyl), or optionally substituted heteroaryl. Pharmaceutically acceptable salts, solvates, hydrates, polymorphs, co-crystals, tautomers, stereoisomers, and isotopically labeled derivatives are also within the scope of the present disclosure.

Examples of nitrogen mustard alkylating agents include, but are not limited to, mustine, cyclophosphamide, chlorambucil, uramustine, ifosfamide, melphalan, and bendamustine. In some embodiments, the nitrogen mustard alkylating agent for use in the methods disclosed herein is a melphalan compound. Melphalan is an alkylating agent of the bischloroethylamine type. As a result, its cytotoxicity appears to be related to the extent of its interstrand cross-linking with DNA, probably by binding at the N7 position of guanine. Like other bifunctional alkylating agents, it is active against both resting and rapidly dividing tumor cells.

Melphalan, also known as sarcolysin, is a chemotherapy drug. The chemical structure of melphalan is shown below.

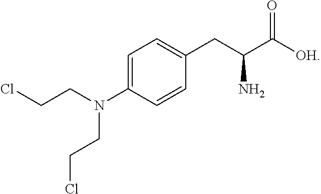 [SMILES: N[C@@H](Cc1ccc(N(CCCl)CCCl)cc1)C(=O)O]

A melphalan compound refers to melphalan, a pharmaceutically acceptable salt or ester thereof, or a derivative thereof. A derivative maintains the core structure noted above and similar alkylating activity, and may include one or more suitable substituents at positions where applicable and where valency permits. Any of the nitrogen mustard alkylating agents disclosed herein (e.g., a melphalan compound such as melphalan) may be mixed with one or more pharmaceutically acceptable carriers, diluents, and/or excipient to form a pharmaceutical composition for administration by a suitable route.

(A) Subjects

A subject as used herein refers to a human or non-human animal. In some instances, the subject is a human patient needs transplantation of hematopoietic cells (e.g., hematopoietic stem cells). Such a subject may be a male or female, and may be of any age group. For example, the subject may be a human adult (e.g., a young adult, a middle-aged adult, or a senior adult). Alternatively, the subject may be a pediatric subject (e.g., an infant, a child, or an adolescent). An infant typically is younger than 12 months old. A child may age from 12 months to 16 years old. An adolescent may age from 10-21 years old. In some instances, the subject is a human patient younger than 2 years. In other examples, the subject is a human patient of 2-6 years old. In further examples, the subject is a human patient of 6-12 years old. Alternatively or in addition, the subject may have a body weight less than 20 kg, for example, less than 15 kg, or less than 10 kg.

The subject may also include any non-human animals including, but not limited to a non-human mammal such as a cynomolgus monkey or a rhesus monkey. In certain embodiments, the non-human animal is a mammal, a primate, a rodent, an avian, an equine, an ovine, a bovine, a caprine, a feline, or a canine. The non-human animal may be a male or a female at any stage of development. The non-human animal may be a transgenic animal or a genetically engineered animal.

In some embodiments, the subject is in need of conditioning prior to hematopoietic cell transplantation (HCT) such as hematopoietic stem cell transplantation (HSCT), which may be autologous or allogeneic. The conditioning may be performed by a reduced intensity conditioning regimen (RIC), e.g., as disclosed herein.

Any of the subjects disclosed herein may be subject to either allogeneic RIC HSCT or autologous RIC HSCT, where the RIC comprises a nitrogen mustard alkylating agent such as a melphalan compound as part of the preparative RIC regimen.

A subject who needs HCT such as HSCT may be a human patient having a malignant disorder. Alternatively, a subject who needs HCT such as HSCT may be a human patient having a non-malignant disorder, e.g., a non-malignant hematologic disease. Examples include, but are not limited to, immune deficiency disorders (e.g., disorders of immune dysregulation), marrow failure disorders, inherited metabolism disorders, anemia, and hemoglobinopathies.

An immune deficiency disorder may be characterized by impairment of the immune system's ability to defend the body against foreign or abnormal cells that invade or attack it (e.g., bacteria, viruses, fungi, and cancer cells). An immune deficiency disorder may also be an autoimmune disorder. In some instances, the immune deficiency disorder may be a primary immune deficiency disorder, which typically is hereditary or genetic. Examples include agammaglobulinemia, ataxia telangiectasia, chronic granulomatous disease, complement deficiencies, DiGeorge syndrome, hemophagocytic lymphohistiocytosis (HLH), hyper IgE syndrome, hyper IgM syndromes, IgG subclass deficiency, innate immune defects NEMO deficiency syndrome, selected IgA or IgM deficiency, combined immune deficiency, severe combined immune deficiency, specific antibody deficiency, transient hypogrammaglobulinemia of infancy, IPEX (Immune dysregulation, polyendocrinopathy, enteropathy, X-linked Syndrome), and WHIM syndrome. In other instances, the immune deficiency disorder is a secondary immune deficiency disorder, which may be caused by environmental factors. Examples include acquired immune deficiency syndrome (AIDS), which may be caused by HIV infection, cancer of the immune system such as leukemia or multiple myeloma, or immune-complex diseases such as viral hepatitis.

Bone marrow failure is characterized by an inability to make enough blood, such as red blood cells, white blood cells, and/or platelets. Marrow failure disorders can be either congenital or acquired.

Inherited metabolic disorders are genetic conditions that result in metabolism problems. Examples include, but are not limited to, familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), porphyria (e.g., erythropoietic protoporphyria), Tay-Sachs disease, or Wilson's disease.

Anemia is a condition characterized by lacking enough healthy red blood cells or hemoglobin. Anemia may be caused by blood loss, decreased or faulty red blood cell production, and/or destruction of red blood cells. Examples include, but are not limited to, sickle cell anemia, iron-deficiency anemia, vitamin-deficiency anemia, bone marrow and stem cell problems (e.g., aplastic anemia, or thalassemia), or anemia associated with other conditions such as advanced kidney disease, hypothyroidism, chronic diseases such as cancer, infection, lupus, diabetes, and rheumatoid arthritis. In some instances, the anemia is inherited, for example, sickle cell anemia or β-thalassemia.

Hemoglobinopathy refers to a group of blood disorders that affect red blood cells. In some instances, hemobinopathy may involve thalassemia syndromes and structural hemoglobin variants (abnormal hemoglobins, e.g., sickle cell disease). α- and β-thalassemia are the main types of thalassemia. The main structural hemoglobin variants are HbS, HbE and HbC.

In other embodiments, the subject may be a human patient who needs the RIC regimen not in association with HCT. Such a subject may be subject to gene therapy.

Alternatively or in addition, the subject disclosed herein may have impaired function of an organ, for example, liver, kidney, intestine (severe colitis), respiratory system, or cardiac system.

(B) Test Dose

The test dose used in the PK studies for predicting a suitable personalized full dose of a nitrogen mustard alkylating agent, such as a melphalan compound, can be about 10-30% of the standard full dose of the nitrogen mustard alkylating agent as used in an RIC regimen for conditioning a subject in association with HCT.

The standard full dose of the nitrogen mustard alkylating agent for a specific subject (e.g., a human patient) in this context would be known to those skilled in the art. For example, a standard full dose of melphalan can range from about 140 to 200 mg/m². The standard full dose of melphalan can be reduced (e.g., by 50% such as 60-90 mg/m², e.g., 70 mg/m²) for patients having radiosensitive disorders. For children (e.g., those having a body weight <10 kg), the standard full dose of melphalan can be 4.7 mg/kg. The standard full dose of melphalan for children having a body weight less than 10 kg with poor tolerance to chemotherapy and/or radiation can be about 2.35 mg/kg.

In some instances, the standard full dose of melphalan can be affected by a subject's kidney function, which, in some instances, can be indicated by the glomerular filtration rate (GFR) of the subject. For example, subjects (e.g., human patients) having a GFR>100 ml/min/1.73 m² may have a reduced standard full dose melphalan of about 70 mg/m² or about 2.3 mg/kg if his or her body weight is less than 12 kg. Alternatively, subjects such as human patients having a GFR<100 ml/min/1.73 m² and ≥60 ml/min/1.73 m² and are >12 kg may have a reduced standard full melphalan dose of about 60 mg/m². Further, subjects such as human patients having a GFR <100 ml/min/1.73 m² and ≥60 ml/min/1.73 m² and are ≤12 kg may have a reduced standard full melphalan dose of about 2 mg/kg.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The test dose for use in the methods disclosed herein may be about 10% to about 30% (e.g., about 10%, about 15%, about 20%, about 25%, or about 30%) of a standard full dose of a nitrogen mustard alkylating agent (e.g., a melphalan compound) for a subject. In some examples, the lowest possible test dose may be selected for robust PK analysis based on population pharmacokinetic simulations using a melphalan PK model. Using the lower limit of quantification of the assay (e.g., 2 ng/mL), the lowest dose that would still give a detectable concentration at least 6 hours post-administration can be selected. In some examples, the test dose is about 10% of a nitrogen mustard alkylating agent (e.g., a melphalan compound) dose of the subject. This test dose would reliably allow the individual PK analysis as disclosed herein and is unlikely to result in measurable biological activity related to the dose.

The test dose of the nitrogen mustard alkylating agent such as a melphalan compound can be given to the subject via a conventional route, for example, intravenous infusion (IV) over a suitable period (e.g., 3-5 minutes). Blood samples can be collected from the subject before and after administration of the test dose. After the administration, biological samples, such as blood samples or urine samples, can be collected at multiple time points.

In some examples, blood samples can be collected at one or more of the following time points after administration of the test dose: at about 5 minutes, about 15 minutes, about 30 minutes, about 45 minutes, about 60 minutes, about 2 hours, about 2.5 hours, about 4 hours, and about 6 hours. In one example, blood samples can be collected at all of these time points post administration of the test dose of the nitrogen mustard alkylating agent such as melphalan.

In some examples, blood samples can be collected at one or more of the time points after administration of the test dose: at about 0.08 hour, 0.5±0.1 hour, 1.5±0.3 hours, and 4.0 hours. In one example, blood samples can be collected at all of these time points post administration of the test dose of the nitrogen mustard alkylating agent, such as melphalan.

In other examples, blood samples can be collected at one or more of the time points after administration of the test dose: between 0.08-0.19 hour, 0.33-0.90 hour, 1.3-2.7 hours, and 3.6-4.0 hours. In one example, blood samples can be collected at all of these time points post administration of the test dose of the nitrogen mustard alkylating agent such as melphalan.

In some embodiments, the RIC regimen applied to a subject comprises a melphalan compound (e.g., melphalan), an antibody specific to CD52 (e.g., alemtuzumab), and a chemotherapeutic (e.g., fludarabine). In some examples, the alemtuzumab and/or the fludarabine can be administered prior to administration of a test dose of the melphalan compound (e.g., melphalan) to the subject. The melphalan PK features of the subject after treatment with alemtuzumab and/or fludarabine can be determined. This may be performed by collecting blood samples at multiple time points after administration of alemtuzumab and/or fludarabine. In other examples, administration of the melphalan compound can be performed before administration of alemtuzumab and/or fludarabine. Multiple blood samples may be collected before and after administration of the melphalan compound. In some instances, some of the blood samples can be collected before administration of alemtuzumab and/or fludarabine and others may be collected after administration of alemtuzumab and/or fludarabine.

In some embodiments, blood samples may be drawn with standard aseptic precautions and the total volume will be limited to 3 ml/kg of patient weight in each 24 hour period. The total volume of blood in any 24 hour period may include blood drawn for clinical testing, research, and discarded samples as required. Exemplary maximum blood volumes for pharmacokinetic studies are outlined in the following Table 1 and Table 2.

Alternatively or in addition, urine samples can be collected from the subject before and after administration of the test dose. The urine samples may be used for measurement of NGAL and KIM-1. Urine samples may be collected prior to the test dose (e.g., within 24 hours), on the day of the test dose, and at one or more time points after the administration of the test dose, e.g., approximately at 8 hours (±2 hours) and at 24 hours (±2 hours) following the end of infusion.

TABLE 1
Maximum Blood Volumes for Infants

| | PK | Total PK |
|---|---|---|
| Age | (ml) | (ml) n = 11 |
| Pre-term newborn | 1 | 11 |
| Term newborn | 1 | 11 |
| 1-24 months | 1 | 11 |

TABLE 2
Maximum Blood Volumes for Children

| | Body Weight | | Total PK | Max Blood Volume |
|---|---|---|---|---|
| Age (yr) | (kg) | PK (ml) | (ml) 11 = 11 | (ml) |
| 2 | 10 | 2 | 22 | 30 |
| | 11 | 2 | 22 | 33 |
| | 12 | 2 | 22 | 36 |
| | 13 | 2 | 22 | 39 |
| 3 | 14 | 2 | 22 | 42 |
| | 15 | 2 | 22 | 45 |
| 4 | 16 | 2 | 22 | 48 |
| | 17 | 2 | 22 | 51 |
| 5 | 18 | 2 | 22 | 54 |
| | 19 | 2 | 22 | 57 |
| 6 | 20 | 2 | 22 | 60 |
| | 21 | 2 | 22 | 63 |
| 7 | 22 | 2 | 22 | 66 |
| | 23 | 2 | 22 | 69 |
| | 24 | 2 | 22 | 72 |
| 8 | 25 | 2 | 22 | 75 |
| | 26 | 2 | 22 | 78 |
| | 27 | 2 | 22 | 81 |

In some instances, approximately 5 ml of urine may be collected in a sterile urine container for each sample. The cumulative volume required can be approximately 15 ml on the day of the test dose (e.g., of melphalan).

The biological samples are subject to PK analysis of the involved the nitrogen mustard alkylating agent such as melphalan as disclosed herein.

(C) Pharmacokinetic (PK) Analysis

The steady-state volume of distribution of melphalan is about 0.5 L/kg. Penetration into cerebrospinal fluid (CSF) is low. The average melphalan binding to plasma proteins is highly variable (range: 53% to 92%). Serum albumin is the major binding protein, accounting for approximately 40% to 60% of the plasma protein binding, while al-acid glycoprotein accounts for about 20% of the plasma protein binding. Approximately 30% of melphalan is (covalently) irreversibly bound to plasma proteins. Interactions with immunoglobulins have been found to be negligible. Melphalan is eliminated from plasma primarily by chemical hydrolysis to monohydroxymelphalan and dihydroxymelphalan (metabolites). Aside from these hydrolysis products, no other melphalan metabolites have been observed in humans. Although the contribution of renal elimination to melphalan clearance appears to be low, one pharmacokinetic study showed a significant positive correlation between the elimination rate constant for melphalan and renal function and a significant negative correlation between renal function and the area under the plasma melphalan concentration/time curve. Adair et al., Cancer Chemother Pharmacol. 17(2):185-8 (1986).

Any of the biological samples disclosed herein may be processed by suitable ways depending upon the assays to use for analyzing the nitrogen mustard alkylating agent such as melphalan or metabolites thereof. For example, when a conventional mass spectrometry method is used, a blood sample can be processed by routine practice (e.g., to obtain plasma) and analyzed, for example, one the same day when the sample is collected. When paper spray mass spectrometry is to be used, no sample processing may be needed since the paper spray method can analyze whole blood samples. Urine samples can be processed using standard protocols.

The biological samples disclosed herein, e.g., blood samples or urine samples, can be subject to suitable assay methods for measuring levels of the nitrogen mustard alkylating agent (e.g., melphalan) or a metabolite thereof (e.g., monohydroxymelphalan and/or dihydroxymelphalan as metabolites for melphalan) in the samples. For example, conventional mass spectrometry may be used to measure the levels of the analytes in the biological samples following conventional methodology. The mass spectrometry analysis may use various types of separation techniques, including, but not limited to, gas chromatography, liquid chromatography mass spectrometry (LS-MS), liquid chromatography tandem mass spectrometry (LS-MS/MS), high performance liquid chromatography mass spectrometry (HPLC-MS), capillary electrophoresis, or ion mobility.

Alternatively, the levels of the nitrogen mustard alkylating agent such as melphalan or metabolites thereof in the biological samples may be determined using paper spray mass spectrometry. Paper spray ionization is a technique used in mass spectrometry to produce ions from a sample to be analyzed. Briefly, a sample (e.g., a blood sample or a urine sample) can be applied to a piece of paper with solvent added. A high voltage can then be applied to create the ions to be analyzed with a mass spectrometer. See, e.g., Liu et al., Analytical Chemistry 82(6):2463-2471 (2010), the relevant disclosures of which are incorporated by reference for the purpose or subject matter referenced herein.

In some embodiments, a biological sample (e.g., blood sample or urine sample) may be analyzed using a TSQ Quantum Ultra mass spectrometer (Thermo Scientific, San Jose, Calif., and USA) interfaced with a paper spray ionization source (Prosolia, Inc. Indianapolis, Ind. USA). Blood samples may be prepared by spiking appropriate melphalan standards and internal standard into drug free human blood. For the paper spray assay, a small amount of blood (12 μL) can first be deposited on paper spray cartridge and after the blood spot has dried, a small volume (ca. 80 μL) of solvent (selected to effectively extract the drug) can be applied to the paper and a high voltage (3-5 kV) can be applied to the paper, inducing an electrospray at the sharp tip of the paper; the solvent evaporates from the droplets generating gas phase ions of the analyte molecules.

In some examples, a paper spray PS-MS/MS assay as disclosed herein can be used for measuring melphalan concentration in whole blood without the need for sample pretreatment or chromatography. In some instances, melphalan can be quantified by using [²H₈]-melphalan as internal standard. Whole blood samples may be obtained from patients receiving melphalan during HSCT at timed intervals post administration to determine each patient's pharmacokinetic profile. The melphalan pharmacokinetics can be determined using WinNonlin v4.0.1 and the area under the curve blood concentration-time profile can be established by linear trapezoidal integration.

The biological samples can be analyzed on the same day as collected. Alternatively, either whole blood samples or plasma samples may be kept at a low temperature (e.g., −70° C.) for storage. Urine samples may be kept in a refrigerator. The samples may be analyzed the next day.

Levels of the nitrogen mustard alkylating agent such as melphalan or metabolites thereof in the biological samples (e.g., whole blood samples, plasma samples, or urine samples) can then be analyzed by compartmental pharmacokinetic analysis, e.g., using a suitable computational software packages such as MW/Pharm (Version 3.82, Mediware, Groningen, the Netherlands) and WinNonlin (Version 4.0.1, Pharsight Corporation, Palo Alto, Calif.) using a Bayesian and weighed least-squares algorithm, respectively. Pharmacokinetic features such as total body clearance, distribution and elimination half-lives, volume of distribution, and area under curve (AUC) can be determined. In some embodiments, AUC can be determined by a conventional method such as the trapezoidal method. See, e.g., Pharmacokinetic and Pharmacodynamic data analysis concepts and applications. 5th Edition. Gabrielson J. Weiner D. Eds. Swedish Pharmaceutical Society. 2016; pp 142-155. The relevant disclosures are incorporated by references for the purpose and subject matter referenced herein.

Optionally, the data may also be analyzed by a population pharmacokinetic approach (NONMEM, version 7.2, GloboMax LLC, Hanover, Md.).

Further, statistical analyses can be performed using conventional approaches. In some examples, R (The R Foundation for Statistical Computing) and JMP (SAS Institute, Inc.) can be used. The results can be reported as descriptive statistics and supplemented wherever possible also by graphical summaries.

(D) Personalized Full Dose Prediction

Pharmacokinetic (PK) features of a subject determined as disclosed herein can be used to predict a suitable personalized full dose of the involved nitrogen mustard alkylating agent such as melphalan. See Example below. In some instances, the suitable personalized full dose of a subject can be predicted based on one or more PK features, for example, total body clearance, distribution and elimination half-lives, volume of distribution, AUC, or a combination thereof. For example, the suitable personalized full dose may be predicted based on total body clearance, which may be median clearance (CL), such as median body weight normalized clearance (CL_{STD}). Alternatively, the suitable personalized full dose may be predicted based on AUC. In some instances, the predicted personalized full dose for a subject (e.g., a subject who needs HCT) may result in a target AUC of about 3.5-6.5 h*μg/ml in the subject based on the AUC of the test dose as determined following the methods disclosed herein. Such a subject may be a human patient having normal organ function.

When the RIC regimen is to be performed not in association with HCT (e.g., in association with gene therapy), the target AUC may be adjusted accordingly (e.g., increased).

In some embodiments, patient characteristics may be taken into consideration, together with the PK features, for predicting suitable personalized full dose of a nitrogen mustard alkylating agent such as melphalan for use in an RIC regimen. Exemplary patient characteristics include, but are not limited to, age, gender, body weight, disease condition, organ function status (e.g., liver function, kidney function, digestive tract function, lung function, cardiac function, or a combination thereof), blood cell count, bone marrow cellularity, infectious status, congenital anomaly, overall clinical status, or a combination thereof. Assessing patient characteristics for determining suitable full dose would be within the knowledge of a skilled person in the pertinent art.

II. Therapeutic Applications

The personalized full dose of a nitrogen mustard alkylating agent, such as a melphalan compound, predicted following the pharmacokinetic studies disclosed herein can be used in a reduced intensity conditioning (RIC) regimen to condition the subject for the needed HCT (e.g., HSCT) therapy, or non-HCT related therapy (e.g., gene therapy).

The RIC regimen disclosed herein involves administering to the subject (e.g., a human patient) who needs HSC transplantation the nitrogen mustard alkylating agent, such as a melphalan compound, at the predicted full dose for that particular subject. In addition to the nitrogen mustard alkylating agent, the RIC may further comprise an antibody specific to CD52 (e.g., alemtuzumab), a chemotherapeutic such as anti-metabolite (e.g., fludarabine), or both. The RIC regimen is expected to put the subject in a good condition for receiving hematopoietic cell (HC), such as hematopoietic stem cell transplantation—to achieve some level of immune suppression such that the transplanted HCs such as HSCs would not be rejected by the host immune system and to reduce side effects associated with myeloablative conditioning regimens commonly used in association with HSC transplantation, particularly HSC transplantation-mediated gene transfer therapy.

As used herein the term “condition” or “conditioning” in the context of a subject pretreatment in need of HC transplantation typically means destroying the bone marrow and immune system of the subject by a suitable procedure, partially or completely. “Myeloablative conditioning” means to destroy bone marrow cells substantially to ablate marrow hematopoiesis and not allow autologous hematologic recovery. “Reduced-intensity conditioning” means to destroy bone marrow cells to some extent such that marrow hematopoiesis is not completely ablated. In some instances, “reduced-intensity conditioning” can be achieved by using less chemotherapy and/or radiation than the standard myeloablative conditioning regimens, for example 50-80% (e.g., 55-75% or 60-70%) of the amount of a chemotherapeutic commonly used for myeloablative conditioning. Additional information of myeloablative conditioning and reduced-intensity conditioning can be found, e.g., in Gyurkocza et al. Blood, 124(3):344-353, 2014, the relevant disclosures of which are incorporated by reference for the purposes or subject matter referenced herein.

Any of the nitrogen mustard alkylating agents disclosed herein (e.g., a melphalan compound, such as melphalan) may be mixed with one or more pharmaceutically acceptable carriers, diluents, and/or excipients to form a pharmaceutical composition for administration by a suitable route. A carrier, diluent, or excipient that is “pharmaceutically acceptable” includes one that is sterile and pyrogen free. Suitable pharmaceutical carriers, diluents, and excipients are well known in the art. The carrier(s) must be “acceptable” in the sense of being compatible with the inhibitor and not deleterious to the recipients thereof. See, e.g., Remington: The Science and Practice of Pharmacy 20th Ed. (2000) Lippincott Williams and Wilkins, Ed. K. E. Hoover.

In some embodiments, the pharmaceutical composition comprising the nitrogen mustard alkylating agent (e.g., a melphalan compound, such as melphalan) may be prepared freshly. For example, the time between reconstitution/dilution and administration of parenteral melphalan may be kept to a minimum (manufacturer recommends completing infusion within <60 minutes) to minimize impact on stability of the agent due to reconstituted and diluted solutions. In some examples, reconstitute 50 mg vial for injection initially with provided 10 mL diluent to yield a 5 mg/mL solution; shake immediately and vigorously to dissolve; immediately dilute the reconstituted solution with NS to a final concentration not to exceed 0.45 mg/m.

A pharmaceutical composition comprising any of the nitrogen mustard alkylating agent, such as a melphalan compound as described herein, may be administered by any administration route known in the art, such as parenteral administration, oral administration, buccal administration, sublingual administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration.

In some embodiments, the pharmaceutical compositions or formulations are for parenteral administration, such as intravenous, intra-arterial, intra-muscular, subcutaneous, or intraperitoneal administration.

Formulations of the nitrogen mustard alkylating agent suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation containing a nitrogen mustard alkylating agent may be suitable for oral, buccal or sublingual administration. Such pharmaceutical compositions may be in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant. Capsules and cartridges (made, for example, from gelatin) for use in an inhaler or insufflator may be formulated to contain a powder mix of the nitrogen mustard alkylating agent and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, the formulations can be pre-loaded in a unit-dose injection device, e.g., a syringe, for intravenous injection.

In one specific example, a pharmaceutical composition comprising melphalan may be administered to the subject by intravenous infusion over 15-30 minutes (e.g., not to exceed 10 mg/min).

The nitrogen mustard alkylating agent such as melphalan at the predicted suitable full dose may be given to a subject by a single dose. If necessary, multiple doses may be given to the subject following routine practice. For example, a subject in need of an HC transplantation may be given a nitrogen mustard alkylating agent (e.g., melphalan) daily, every 2 days, every 3 days, or longer, prior to receiving the HC transplantation.

After or currently with reduced-intensity conditioning, HC such as HSC transplantation may be administered to the subject via a routine procedure (e.g., infusion). hematopoietic cells (HCs) refer to any cells having hematopoietic origin, include those lodged within the bone marrow (e.g., HSCs), cells differentiated therefrom (for example, those circulating in the blood such as red blood cells, white blood cells, and platelets), HCs such as HSCs derived from in vitro differentiation of stem cells (e.g., induced pluripotent stem cells or iPSCs).

Hematopoietic stem cell transplantation (HSCT) is the transplantation of multipotent hematopoietic stem cells, which may be derived from bone marrow, peripheral blood, umbilical cord blood, or from iPSCs. HCs can be obtained using conventional methods. For example, HCs can be isolated from bone marrow, peripheral blood cells, and/or umbilical cord blood. One or more mobilizing agents, such as Plexifor, may be used to increase the availability of HCs. Alternatively, the HCs can be derived from stem cells (e.g., induced pluripotent stem cells which can be differentiated from somatic cells such as skin cells). The HCs can be cultured ex vivo prior to transplantation to a subject.

In some embodiments, the HCs may be isolated from the same subject (autologous), cultured ex vivo when needed, and be transplanted back to the subject. Administration of autologous cells to a subject may result in reduced rejection of the stem cells as compared to administration of non-autologous cells. Alternatively, the HCs can be allogenic, i.e., obtained from a different subject of the same species. For allogeneic HC transplantation, allogeneic HCs may have a HLA type that matches with the recipient.

In any of the HC transplantation therapies described herein, suitable HCs such as HSCs can be collected from the ex vivo culturing method described herein and mixed with a pharmaceutically acceptable carrier to form a pharmaceutical composition, which is also within the scope of the present disclosure.

In some instances, when applicable the transplanted cells may be modified to deliver a therapeutic effect. For example, but in no way defining or limiting, such cells may be genetically engineered cells to contain a gene to encode for a protein which the subject was previously deficient because of a mutation in his/her own genetic makeup. In other instances, the cells may contain a gene which is modified to express for increased amounts of a protein to counteract or offset another protein or product in the subject. In some instances, this may be accomplished by transducing the cells with a viral vector. A “vector”, as used herein is any vehicle capable of facilitating the transfer of genetic material (e.g., a shRNA, siRNA, ribozyme, antisense oligonucleotide, protein, peptide, or antibody) to a cell in the subject, such as HCs. In general, vectors include, but are not limited to, plasmids, phagemids, viruses, and other vehicles derived from viral or bacterial sources that have been manipulated by the insertion or incorporation of a sequence encoding a gene of interest. Viral vectors include, but are not limited to nucleic acid sequences from the following viruses: retrovirus; lentivirus; adenovirus; adeno-associated virus; SV40-type viruses; polyoma viruses; Epstein-Barr viruses; papilloma viruses; herpes virus; vaccinia virus; polio virus. One can readily employ other vectors not named but known to the art.

Viral vectors may be based on non-cytopathic eukaryotic viruses in which nonessential genes have been replaced with a sequence encoding a gene of interest. Non-cytopathic viruses include retroviruses (e.g., lentivirus, gamma-retrovirus, or foamy virus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known in the art.

Other viral vectors include adeno-viruses and adeno-associated viruses, which are double-stranded DNA viruses that have also been approved for human use in gene therapy. The adeno-associated virus can be engineered to be replication deficient and is capable of infecting a wide range of cell types and species.

Other vectors include plasmid vectors. Plasmid vectors have been extensively described in the art and are well known to those of skill in the art. See, e.g., Sambrook et al. Molecular Cloning: A Laboratory Manual. Cold Spring Harbor Laboratory Press; 4th edition (Jun. 15, 2012). Exemplary plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA, such as a sequence encoding a γ-globin gene.

In some examples, the HSCs described herein (e.g., human adult HSCs) can be genetically engineered to express a gene of interest suitable for treatment of a target disease, for example, a γ-globin for use in treating anemia, such as sickle cell anemia and thalassemia. See, e.g., US 20110294114 and WO 2015/117027, the relevant teachings of each of which are incorporated by reference for the purposes or subject matter referenced herein.

Any of the HC cells disclosed herein may be administered to a subject who has undergone or is undergoing the reduced-intensity conditioning regimen as disclosed herein via a suitable route, for example, intravenous infusion. In some embodiments, the subject may be given at least 10⁵ cells per infusion, for example, at least 10⁶, at least 10⁷, or at least 10⁸ cells. Typically, HC transplantation would be carried out after the reduced-intensity conditioning so as to give time for the host HCs to be inhibited or eliminated by the nitrogen mustard alkylating agent. The HC cells may be given to a subject 12 hours after the reduced-intensity conditioning, 24 hours after the reduced-intensity conditioning, 36 hours after the reduced-intensity conditioning, 48 hours after the reduced-intensity conditioning, 72 hours after the reduced-intensity conditioning, one week after the reduced-intensity conditioning, or longer.

In some embodiments, the HC transplantation can be co-used with a therapeutic agent for a target disease, such as those described herein. The efficacy of the stem cell therapy described herein may be assessed by any method known in the art and would be evident to a skilled medical professional. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject.

The methods disclosed herein, involving any of the reduced-intensity conditioning regimens disclosed herein followed by hematopoietic cell transplantation also disclosed herein can be used for treating suitable target diseases, particularly those that require gene transfer therapy.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

The subject to be treated by the methods described herein can be a human (e.g., a male or a female of any age group). In some instances, the subject can be a pediatric subject (e.g., an infant, child, or an adolescent) or an adult subject (e.g., a young adult, a middle-aged adult, or a senior adult).

In some embodiments, the subject (e.g., a human subject), may have, be suspected of having, be at risk of having, or be predisposed to having a disease that can be treated by gene transfer therapy, for example, a genetic disorder. In some instances, the subject is a human patient having a hemoglobinopathy, which refers to a disorder associated with a genetic defect that results in abnormal structure of one of the globin polypeptide of hemoglobin or reduction of the globin polypeptide, e.g., alpha- (α-), beta- (β-), or gamma- (γ-) globin. Common hemoglobinopathies include sickle-cell disease and thalassemia such as β-thalassemia. In some instances, the subject is a human patient having anemia, such as sickle-cell anemia, congenital dyserythropoietic anemia, and thalassemia such as β-thalassemia.

In specific examples, the methods described herein aim at treating sickle cell disease (SCD). SCD affects the β-globin gene and is one of the most common genetic defects, resulting in the production of a defective sickle-globin (HbS, comprised of two normal α-globin and two β/sickle-globin molecules). HbS polymerizes upon deoxygenation and changes the shape of discoid red blood cells (RBCs) to bizarre sickle/hook shapes. Sickled RBCs clog the microvasculature, causing painful acute organ ischemic events and chronic organ damage that foreshortens the life span of SCD patients to 45 years. This disease affects over 110,000 Americans, with 1000 newborns with SCD born every year and nearly 1000 babies born with this disease annually in Africa.

Fetal hemoglobin (HbF, comprised of α and γ globins, α₂γ₂) is produced during the fetal life and the first 6-9 months of age and has strong anti-sickling properties and protects the infant from sickling in the first year of life. Indeed, individuals with hereditary persistence of HbF that have SCD are asymptomatic. Hydroxyurea, a chemotherapeutic drug that increases HbF, is FDA-approved for ameliorating symptoms of SCD. However, hydroxyurea does not work for all patients, and due to daily life-long intake, is associated with poor compliance. Hence, better therapeutic options are needed for SCD.

In some embodiments, the HSCs used in the methods described herein are genetically modified to express a γ-globin, which can form HbF in a recipient of the HSCs, who can subject to the reduced-intensity conditioning before the transplant.

The γ-globin protein may be of any suitable species, for example, human, monkey, chimpanzee, pig, mouse, rat, etc. In some instances, the γ-globin protein may be a wild-type protein. In others, the γ-globin protein may be a mutated form of a wild-type γ-globin protein, which retains substantially similar bioactivity as the wild-type counterpart and may have an increased binding affinity to the α-globin subunit, thereby forming fetal hemoglobin (α₂γ₂) at a high level so as to compete against the defective adult hemoglobin (α₂γ₂, in which the n-chain is defective). Such a γ-globin mutant may comprise a substitution at position 17 of a wild-type counterpart (e.g., a G→D substitution). In some instances, the γ-globin mutant contains a substitution at position 17 of a wild-type counterpart and share a sequence homology of at least 85% (e.g., at least 90%, at least 95%, at least 97%, at least 98% or above) relative to the wild-type counterpart.

Other exemplary γ-globin proteins are well known in the art and can be retrieved from publically available gene database such as GenBank, using the above-noted sequences as queries. Examples include GenBank Accession nos. P02099.2, NP_001164974.1, and NP_001040611.2.

In addition, the treatment methods disclosed herein may target a malignant disorder, which can be treated by HCT. Alternatively, the methods may target a non-malignant disorder, e.g., a non-malignant hematologic disease, such as those disclosed herein. Examples include, but are not limited to, immune deficiency disorders (e.g., disorders of immune dysregulation), bone marrow failure, inherited metabolism disorders, anemia, and hemoglobinopathies.

Where it is desirable, the subject can further receive a second HC transplantation after the transplantation of the first population of HCs. The second HC transplantation can be performed any time after the first HC transplantation. For example, the second HC transplantation can be performed about 3 days or longer, including 4 days, 5 days, 6 days, 7 days, 2 weeks, 3 weeks, 4 weeks, or longer, after the first HC transplantation.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. 1985); Transcription and Translation (B. D. Hames & S. J. Higgins, eds. 1984); Animal Cell Culture (R. I. Freshney, ed. 1986); Immobilized Cells and Enzymes (IRL Press, 1986); and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Optimization of Melphalan Doses Based on Test Dose Melphalan Pharmacokinetics in Human Subjects Undergoing Reduced Intensity Conditioning Allogenic Hematopoietic Cell Transplantation for Non-Malignant Disorders

High dose melphalan (HDM) is an important component of reduced intensity conditioning (RIC) regimens in children and young adults undergoing allogeneic hematopoietic cell transplantation (HCT) for non-malignant disorders and can be associated with significant non-hematological toxicity. Reported herein is a study on melphalan pharmacokinetics (PK) in children and young adults undergoing allogeneic HCT for non-malignant disorders using a uniform combination of fludarabine, melphalan and alemtuzumab based RIC regimen. Feasibility of melphalan test dose PK guided precision dosing was also evaluated using a novel paper spray mass spectrometry assay (PS-MS/MS) and conventional liquid chromatography electrospray ionization mass spectrometry (LC-MS/MS).

### Methods

(i) Pharmacokinetic Studies of Patients Receiving Test Doses and/or Full Doses

Patients undergoing allogeneic HCT for non-malignant disorders with a uniform RIC regimen that included alemtuzumab, fludarabine and melphalan were enrolled in this study. Full standard dose of melphalan was set as follows:
140 mg/m² in patients having a body weight >10 kg,
4.7 mg/kg in patients having a body weight <10 kg, and
70 mg/m² in one patient with a radio sensitivity disorder.

The patients were given a test dose of melphalan (10% of the standard full dose) prior to start of their preparative regimen. Test dose of 10% of the full standard dose was determined to be the lowest dose level that would be measurable for a sufficiently long time interval (0-4h) to allow reliable AUC estimation. Accordingly, the test doses used in this study were 14 mg/m², 7 mg/m² and 0.47 mg/kg for patients having the standard full doses of 140 mg/m², 70 mg/m² and 4.7 mg/kg, respectively.

Blood samples were obtained for PK measurement after the administration of test dose and the full standard dose of melphalan. A total of 10 blood samples were obtained around each dose of melphalan: at baseline (5-10 min prior to start of the melphalan infusion of) and then approximately at 5 min, 15 min, 30 min, 45 min, 60 min, 2 hour, 2.5, 4, and 6 hours after the end of the melphalan infusion. Samples were collected on ice and transported to the institution's mass spectrometry laboratory for instant analysis by PS-MS/MS and conventional LC-MS/MS. Patient data collected for analysis included baseline organ function, presence of oral and gastrointestinal mucositis, renal and liver dysfunction including VOD, initial donor chimerism, graft rejection, and acute and chronic GVHD (aGVHD and cGVHD). The Common Terminology Criteria for Adverse Events (CTCAE) version 4.0 was used to evaluate adverse events. Acute and chronic GVHD were assessed by standardized published criteria. See, e.g., Glucksberg H, et al. Transplantation. 1974; 18:295-304 and Filipovich A H, et al. Biol Blood Marrow Transplant. 2005; 11:945-956. Interim analyses were completed after enrollment of 5 patients and 10 patients respectively to validate data following 25% and 50% of intended total patient enrollment. The AUC range that led to full donor chimerism without excess toxicity in majority of patients was selected to be the desired target AUC for full dose adjustment. In patients with baseline impaired organ function, personalized full dose melphalan was adjusted to limit toxicity if test dose PK predicted standard full dose AUC was higher than the desired target AUC range.

### (ii) Assay Methodology

Melphalan levels were measured by validated PS-MS/MS and LC-MS\MS methods (Mirkou A, etc., J Chromatogr B Analyt Technol Biomed Life Sci. 2009; 877:3089-3096). For the PS-MS/MS method, all samples were analyzed using an automated Paper Spray ion source (Prosolia, Inc. Indianapolis, Ind.) interfaced with a TSQ Quantum Ultra mass spectrometer (Thermo Scientific, San Jose, Calif.) operated in selected reaction monitoring (SRM) mode. XCalibur software was used to control the MS and to process data. Specific precursor/product ions were simultaneously monitored for melphalan and its stable isotopically-labelled internal standard (D8-melphalan) by collision-induced dissociation (CID). Blood samples were prepared by spiking known amounts of melphalan standards and the internal standard into drug free human blood to establish a calibration curve and quality control samples. A small amount of patient's blood (124) equilibrated with D8-melphalan solution was deposited on the Paper Spray cartridge and dried. A small volume of solvent optimized to efficiently extract the drug was applied to the paper. A stepwise high voltage (3-5 kV) was then applied inducing an electrospray ionization at the tip of the paper; the solvent evaporates from the droplets generating gas phase ions of the analyte, which then can be detected by a mass spectrometer. The analysis time for each sample was about 3 minutes with essentially no prior sample preparation. Plasma samples were obtained by centrifugation at 3000 rpm for 15 min and then store at −80° C. degree until analysis by LC-MS\MS. Plasma samples were also obtained and stored at −80° C. for subsequent analysis by PS-MS/MS, when it was not possible to do whole blood rapid PS-MS/MS (i.e. due to equipment malfunction or non-availability of paper spray cartridges). The lower limit of quantitation (LLOQ) was 25 ng/mL for PS-MS/MS and 2 ng/mL for LC-MS/MS. Intra-day and inter-day precision (variability as CV %) was 15%.

(iii) Data Analysis

Plasma concentration data were analyzed by Bayesian analysis with the software package MW/Pharm (Version 3.82, Mediware, Prague, Czech Republic) using a published population PK model (Mizuno K, etc., Clin Pharmacokinet. 2018; 57:625-636). Individual parameter estimates generated using Bayesian algorithm included clearance normalized by allometrically scaled body weight of 70 kg, elimination half-life, volume of distribution and AUC. The data were also analyzed by a population pharmacokinetic approach (NONMEM, version 7.2, GloboMax LLC, Hanover, Md.). Data visualization and statistical analyses were performed using R (The R Foundation for Statistical Computing) and GraphPad Prism 8 (GraphPad Software, San Diego, Calif.). The results are reported as descriptive statistics and supplemented wherever possible also by graphical summaries and regression equations describing the relationships. Prediction error was calculated as follows:

$$Prediction error \% = \frac{\begin{matrix}Observed full dose AUC - \\ Test dose PK predicted full dose AUC\end{matrix}}{Observed full dose AUC}$$

### Results

### (i) Patient Demographics

Twenty-six patients undergoing allogeneic HCT using RIC containing alemtuzumab, fludarabine and melphalan were enrolled in the study. Median age of the group was 2.6 years (range: 0.3-25.8 years). There were 18 males and 8 females. Patient and transplant characteristics including indication for HCT are shown in Table 3. Two patients with sickle cell disease (n=2) were excluded from the analysis due to renal hyperfiltration associated with SCD which we believed would confound PK results. Data were analyzed in 24 patients. Of these, 22 patients received a melphalan test dose (2 parents declined a melphalan test dose after enrollment). Twenty three patients receive standard full dose melphalan, one patient died prior to administration of full dose melphalan. Of the 23 patients who received full dose melphalan, 17 patients received standard full dose melphalan (140 mg/m² or 4.7 mg/kg in patients <10 kg), or 70 mg/m² (n=1, which is a standard 50% dose reduction for a radiosensitive disorder); and 6 patients with baseline organ dysfunction received an adjusted full dose of melphalan based on test dose PK. One patient underwent transplant twice and received a melphalan test dose on both occasions. Individual melphalan test dose and full dose PK characteristics are shown in Table 4. Laboratory data and PK parameter estimates for all 24 patients and patients categorized by standard full dose and adjusted full dose are shown in Table 5. Median GFR was 117 ml/min/1.73 m² (range, 55-216 ml/min/1.73 m²).

TABLE 3
Patient Demographics and Transplant Characteristics

| Characteristics | Number or Median (Range) | |
|---|---|---|
| Number of patients | 26 | |
| Age (years) | 2.6 | (0.3-25.8) |
| Body Weight (kg) | 0.59 | (0.25-2.1) |
| Body Surface Area (m²) | 14.8 | (3.7-96.3) |
| Height (cm) | 88.8 | (51.2-181.0) |

Diagnosis

| Hemophagocytic Lymphohistiocytosis | 10 | |
|---|---|---|
| Severe Combined Immune Deficiency | 5 | |
| Combined Immune Deficiency | 3 | |
| Aplastic Anemia/Bone Marrow Failure | 3 | |
| Sickle Cell Disease | 2 | |
| IPEX/IPEX-like | 2 | |
| Erythropoietic Protoporphyria | 1 | |
| BUN | 12 | (4.0-39.0) |
| Serum creatinine (mg/dL) | 0.27 | (0.14-0.88) |
| GFR (mL/min/1.73 m²) | 117 | (24-239) |

TABLE 4
Individual melphalan test dose and full dose pharmacokinetic features

| | | DEMOGRAPHICS | | | | TEST DOSE PK | | |
|---|---|---|---|---|---|---|---|---|
| | FullDose | | | | GFR | TDOSE | | TAUC |
| [symbol] TUDY | (Standard/ | | AGE | WT | [symbol] mL/min/ | (mg/m2 or | TDOSE | mg · |
| ID | Adjusted) | SEX | (years) | (kg) | 1.73 sqm [symbol] | mg/kg) | mg | h/L |
| MEL-2 | Standard | M | 5.6 | 22.5 | 188 | 14 mg/m2 | 5.8 | 0.29 |
| MEL-3 | Standard | M | 2.6 | 12.0 | 132 | 7 mg/m2 | 7.6 | 0.65 |
| MEL-4 | Standard | F | 16.7 | 96.3 | 152 | 14 mg/m2 | 29.3 | 0.59 |
| MEL-5 | Standard | M | 1.5 | 9.4 | NA | NA | NA | NA |
| MEL-7 | Standard | F | 5.3 | 13.4 | 74 | 14 mg/m2 | 8.3 | 0.51 |
| MEL-10 | Standard | F | 4.2 | 14.8 | 142 | 14 mg/m2 | 8.4 | 0.52 |
| MEL-11 | Standard | F | 3.4 | 19.9 | 239 | 14 mg/m2 | 10.6 | 0.57 |
| MEL-12 | Standard | F | 1.6 | 15.0 | 113 | 0.47 mg/kg | 8.1 | 0.52 |
| MEL-13 | Adjusted | M | 1.1 | 5.4 | 29 | 0.24 mg/kg | 1.3 | 0.70 |
| MEL-16 | Standard | M | 0.3 | 6.5 | NA | NA | NA | NA |
| MEL-17 | Standard | M | 4.2 | 35.4 | 164 | 14 mg/m2 | 14.0 | 0.52 |
| MEL-18 | Adjusted | F | 12.8 | 17.5 | 37 | 14 mg/m2 | 9.5 | 1.45 |
| MEL-19 | Standard | M | 2.3 | 22.0 | 143 | 14 mg/m2 | 10.0 | 0.72 |
| MEL-20 | Standard | F | 0.3 | 3.7 | 71 | 0.47 mg/kg | 1.7 | 0.56 |
| MEL-22 | Standard | F | 0.3 | 4.0 | 117 | 0.47 mg/kg | 1.9 | 0.48 |
| MEL-23 | Standard | M | 0.5 | 4.5 | 116 | 0.47 mg/kg | 2.1 | 0.61 |
| MEL-24 | Standard | M | 0.8 | 7.7 | 95 | 0.47 mg/kg | 3.4 | 0.55 |
| MEL-25 | Adjusted | M | 0.7 | 5.5 | 24 | 0.47 mg/kg | 2.4 | 1.16 |
| MEL-26 | Standard | M | 0.3 | 5.5 | 89 | 0.47 mg/kg | 2.6 | 1.11 |
| MEL-28 | Adjusted | M | 0.9 | 7.1 | 33 | 0.24 mg/kg | 1.7 | 0.84 |
| MEL-29 | Adjusted | M | 25.8 | 80.6 | 120 | 14 mg/m2 | 28.7 | 0.71 |
| MEL-30 | Standard | M | 12.8 | 32.5 | 180 | 14 mg/m2 | 15.7 | 0.57 |
| MEL-31 | Adjusted | M | 16.9 | 62.4 | 84 | 14 mg/m2 | 24.2 | 1.38 |
| MEL-32 | Standard | M | 16.4 | 49.1 | 139 | 14 mg/m2 | 22.4 | 0.44 |
| MEL-35 | Adjusted | M | 18.0 | 81.7 | 43 | 14 mg/m2 | 27.2 | 0.74 |
| | | TEST | EVALU- | | | | | EVALU- |
| | | DOSE PK | ATION | FULL DOSE PK | | | | ATION |
| | | TCLstd | Predicted | FDOSE | | | | Predicted |
| | [symbol] TUDY | L/h/ | FAUC | (mg/m2 or | FDOSE | FAUC | FCLstd | FAUC |
| | ID | 70 kg | mg · h/L | mg/kg [symbol] | mg | mg · h/L | L/h/70 kg | mg · h/L |
| | MEL-2 | 47.3 | 2.9 | 140 mg/m2 | 58 | 5.0 | 27.4 | 2.9 |
| | MEL-3 | 44.0 | 6.5 | 70 mg/m2 | 76 | 6.1 | 46.9 | 6.5 |
| | MEL-4 | 38.8 | 5.9 | 140 mg/m2 | 293 | 5.4 | 43.1 | 5.9 |
| | MEL-5 | NA | NA | 4.7 mg/kg | 44 | 3.0 | 66.8 | NA |
| | MEL-7 | 56.2 | 5.1 | 140 mg/m2 | 83 | 9.5 | 30.2 | 5.1 |
| | MEL-10 | 52.2 | 5.2 | 140 mg/m2 | 84 | 4.6 | 59.4 | 5.2 |
| | MEL-11 | 47.8 | 5.7 | 140 mg/m2 | 100 | 4.1 | 62.8 | 5.4 |
| | MEL-12 | 49.5 | 5.2 | 4.7 mg/kg | 81 | 6.6 | 38.9 | 5.2 |
| | MEL-13 | 12.1 | 14.0 | 1.8 mg/kg | 9.7 | 3.6 | 18.6 | 5.5 |
| | MEL-16 | NA | NA | 4.7 mg/kg | 31 | 4.8 | 38.3 | NA |
| | MEL-17 | 45.2 | 5.2 | 140 mg/m2 | 140 | 5.5 | 42.7 | [symbol] |
| | MEL-18 | 18.5 | 14.5 | 140 mg/m2 | 36 | 5.4 | 18.8 | [symbol] |
| | MEL-19 | 33.1 | 7.2 | 140 mg/m2 | 100 | 6.2 | 38.3 | [symbol] |
| | MEL-20 | 27.5 | 5.6 | NA | NA | NA | NA | [symbol] |
| | MEL-22 | 33.9 | 4.8 | 4.7 mg/kg | 18.8 | 4.8 | 33.9 | [symbol] |
| | MEL-23 | 27.0 | 6.1 | 4.7 mg/kg | 21 | 4.3 | 38.0 | [symbol] |
| | MEL-24 | 32.3 | 5.5 | 4.7 mg/kg | 34 | 6.0 | 29.4 | [symbol] |
| | MEL-25 | 13.6 | 11.6 | 0.4 mg/kg | 2.4 | 1.0 | 15.1 | [symbol] |
| | MEL-26 | 15.7 | 11.1 | 4.7 mg/kg | 25.6 | 6.9 | 25.0 | [symbol] |
| | MEL-28 | 11.3 | 16.8 | 1.2 mg/kg | 8.5 | 3.9 | 12.3 | [symbol] |
| | MEL-29 | 35.9 | 7.1 | 100 mg/m2 | 200 | 4.1 | 43.5 | [symbol] |
| | MEL-30 | 49.6 | 5.7 | 140 mg/m2 | 140 | 6.8 | 36.9 | [symbol] |
| | MEL-31 | 19.2 | 13.8 | 41 mg/m2 | 71 | 3.9 | 19.7 | [symbol] |
| | MEL-32 | 67.1 | 4.4 | 140 mg/m2 | 224 | 5.6 | 50.6 | [symbol] |
| | MEL-35 | 32.8 | 7.4 | 100 mg/m2 | 200 | 5.0 | 34.5 | [symbol] |

T: weight, GFR: glomerular filtration rate, PK: Pharmacokinetics, TDOSE: test dose; TAUC: test dose AUC, TCLstd: test dose body weight normalized clearance by allometric scaling, FDOSE: full dose, FAUC: full dose AUC, FCLstd: body weight normalized clearance by allometric scaling. MEL-25 and MEL-28 are the same patient (underwent transplant twice). [symbol]
indicates data missing or illegible when filed

TABLE 5
Laboratory data and PK parameter estimates in 24 patients
receiving test dose and/or full dose

| | | | | | PK-adjusted full | |
|---|---|---|---|---|---|---|
| | Total | | Standard full | | dose*² | |
| Number of patients | N = 24 (25)*¹ | | N = 17 (17) | | N = 6 (7) | |
| (transplants) | | | | | | |
| Male/Female | 16/8 | | 11/6 | | 6/1 | |
| Age (years) | 2.6 | (0.3-25.8) | 2.5 | (0.3-16.7) | 12.8 | (0.7-25.8) |
| Body weight (kg) | 14.8 | (3.7-96.3) | 14.8 | (4.0-96.3) | 17.5 | (5.4-84.3) |
| GFR (ml/min/1.73 m²) | 117 | (24.0-239) | 139 | (74-239) | 37.0 | (24.0-120)** |
| BUN (mg/dL) | 12.0 | (4.0-39.0) | 11.0 | (40-20.0) | 25.0 | (9.0-39.0)*** |
| ALB (g/dL) | 3.2 | (2.5-4.0) | 3.2 | (2.6-4.0) | 3.2 | (2.5-3.8) |
| Hb (g/dL) | 9.7 | (6.4-14.1) | 9.7 | (6.4-13.1) | 9.5 | (7.2-14.1) |
| HCT (%) | 29.4 | (16.6-41.6) | 29.4 | (16.6-37.9) | 28.6 | (21.5-41.6) |
| Test dose PK | N = 22 (23) | | N = 16 (16) | | N = 6 (7) | |
| Test dose | | | | | | |
| Patients ≥10 kg: Dose (mg/m²) | 14.0 | (7.0-14.3) | 14.0 | (7.0-14.3) | 14.0 | (13.7-14.0) |
| Patients <10 kg: Dose (mg/kg) | 0.45 | (0.23-0.48) | 0.47 | (0.44-0.48) | 0.24 | (0.23-0.44)* |
| Test dose CL (L/h/70 kg) | 33.9 | (11.3-67.2) | 18.5 | (11.3-35.9) | 45.2 | (15.7-67.1)*** |
| Test dose AUC (h * μg/mL) | 0.59 | (0.29-1.45) | 0.55 | (0.29-1.11) | 0.84 | (0.70-1.45)*** |
| Test dose PK predicted full | 5.9 | (11.3-67.2) | 5.7 | (4.3-11.1) | 13.8 | (7.1-16.4)*** |
| close AUC (h * μg/mL) | | | | | | |
| Full dose PK | N = 23 (24) | | N = 17 (17) | | N = 6 (7) | |
| Full dose | | | | | | |
| Patients ≥10 kg: Dose (mg/m²) | 135 | (40.9-143) | 140 | (69.6-143) | 75.9 | (40.9-99.4)*** |
| Patients <10 kg: Dose (mg/kg) | 4.65 | (0.44-4.78) | 4.68 | (4.42-4.78) | 1.20 | (0.44-1.80)*** |
| Full dose CL (L/h/70 kg) | 37.4 | (12.3-66.8) | 38.3 | (25.0-66.8) | 18.8 | (12.3-43.5)** |
| Full dose AUC (h * μg/mL) | 5.0 | (1.0-9.5) | 5.5 | (3.0-9.5) | 3.9 | (1.0-5.4)* |
| Distribution of AUC (h * μg/mL) | | | | | | |
| <3.5 | 8.3% | (2/24) | 5.9% | (1/17) | 14.3% | (1/7) |
| 3.5-≤6.5 | 75.0% | (18/24) | 70.6% | (12/17) | 85.7% | (6/7) |
| >6.5 | 16.7% | (4/24) | 23.5% | (4/17) | 0.0% | (0/7) |

*¹A total of 21 patients (22 transplants) received both test and full dose. The median with minimum and maximum values were calculated based on the number of transplants.
*²Statistical analysis was performed using unpaired t-test to compare the data between patients receiving standard full dose vs PK-adjusted full dose.
*p < 0.5,
**p < 0.01,
***p < 0.001

### (ii) Characterization of Melphalan Test Dose PK

Twenty-two patients undergoing 23 transplants received a melphalan test dose. Test dose was 14 mg/m² (i.e. 10% of 140 mg/m2) in 14 transplants, 0.47 mg/kg (i.e. 10% of anticipated full dose of 4.7 mg/kg) in 5 transplants, 0.24 mg/kg (10% of anticipated full dose of 2.35 mg/kg) in 2 transplants and 7 mg/m² (10% of anticipated full dose of 70 mg/m²) in one transplant. Median test dose AUC for all patients was 0.6 h*μg/mL (range, 0.29-1.45 h*μg/mL). Similarly, median clearance for test dose melphalan was 33.9 L/h/70 kg (range, 11.3-67.2 L/h/70 kg). In 16 patients, liver and renal function tests were within normal range for age with GFR >70 ml/min/1.73 m². In this group with normal organ function, median test dose AUC was 0.55 h*μg/mL (range, 0.29-1.1 h*μg/mL) and median test dose clearance was 44.6 (range, 15.7-67.2 L/h/70 kg). Median predicted AUC for standard full dose melphalan in these patients was 5.5 h*μg/mL (range, 2.9-11.1 h*μg/mL).

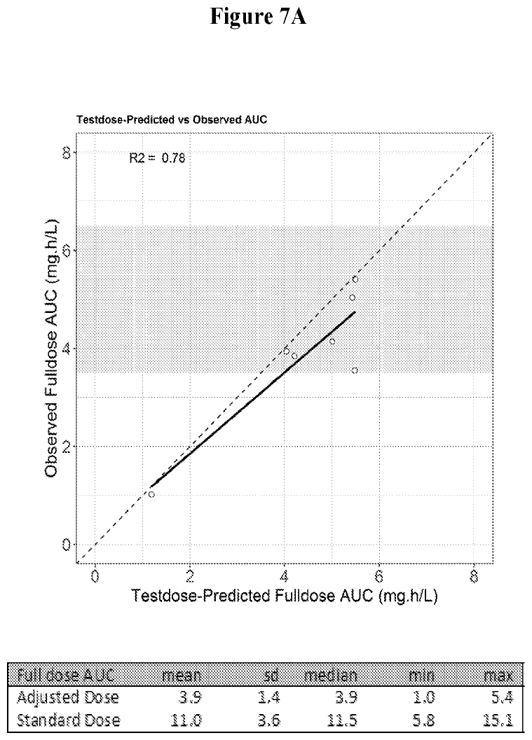

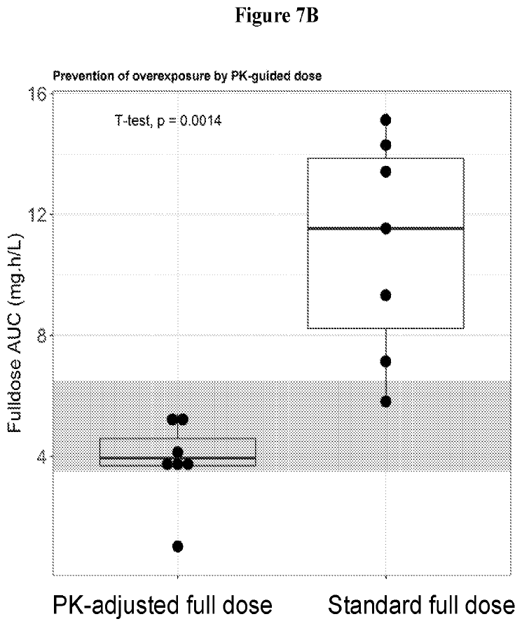

In patients with normal organ function, we further compared test dose clearance and AUC in patients <10 kg and patients >10 kg as their melphalan dosing was different (0.47 mg/kg vs 14 mg/m²). Clearance was lower in patients <10 kg compared to patients >10 kg, but test dose AUC was similar in both groups. In patients >10 kg with normal organ function (n=11, dose 14 mg/m²), median test dose clearance was 47.8 L/h/70 kg (range 33.1-67.2 L/hr./70 kg) and median test dose AUC was 0.52 h*μg/mL (range, 0.29-0.72 h*μg/mL) as shown in supplemental FIG. 7A. In patients <10 kg with normal organ function (n=5, dose 0.47 mg/kg), median test dose clearance was lower at 27.6 L/hr./70 kg (range 15.7-34.0 L/h/70 kg) as shown in FIG. 7B, but median test dose AUC was similar to patients >10 kg at 0.56 h*μg/mL (range, 0.48-1.11 h*μg/mL). A 4-month infant in particular had considerably lower clearance (15.7 L/h/70 kg) and higher test dose predicted full dose AUC (11.1 h*μg/mL) despite normal renal and liver function for age.

Importantly, test dose PK was utilized to dose adjust full dose in patients with organ impairment

(iii) Characterization of Standard Full Dose Melphalan PK

Twenty three patients received melphalan full dose. Of these, 17 patients received standard full dose; 140 mg/m2 (n=10) or 4.7 mg/kg in patients weighing <10 kg (n=6) or 70 mg/m2 (n=1, which is a standard 50% dose reduction for a radiosensitive disorder). Median GFR was 117 ml/min/1.73m2 (range, 55-216 ml/min/1.73m2). In these 17 patients who received standard full dose, median AUC was 5.5 h*ug/mL (range, 3.0-9.5 h*ug/mL). Median clearance was 38.3 L/h/70 kg (range, 25.0-66.8 L/h/70 kg). Median AUC was 5.2 h*ug/mL (range: 3.5 and 6.5 h*ug/mL) in 12/17 (70.6%) of patients. Median AUC in patients weighing <10 kg who received the standard dose of 4.7 mg/kg (n=6) was 4.8 h*ug/mL (range 3.0-6.9 h*ug/mL) and median AUC in patients weighing >10 kg who received standard dose of 140 mg/m2 (n=11) was 5.5 h*ug/mL (range 4.1-9.5 h*ug/mL).

None of the patients who received standard dose melphalan developed primary graft failure and all engrafted with full donor chimerism (>95%). Gastrointestinal mucositis was the most common side effect of the conditioning regimen. Ten patients developed grade 3 mucositis, 5 patients developed grade 2 mucositis, one patient each developed grade 1 and grade 4 mucositis. Notably, the patient with grade 4 mucositis developed gastrointestinal bleeding and had the highest melphalan exposure (AUC 9.5 h*ug/mL) in our study. Two patients developed VOD of the liver including one patient who also developed diffuse alveolar hemorrhage, whose full dose melphalan AUC was 5.0 h*ug/mL, similar to median AUC of this group. This patient had ataxia telangiectasia, a radiosensitive disorder and excess liver iron (18,000 micrograms/gm of liver tissue). The second patient who developed VOD was a 4-month-old infant whose full dose melphalan AUC was higher at 6.9 h*ug/mL, compared to other patients who received standard dose melphalan. Four patients (15%) developed GVHD and risk of GVHD did not differ by melphalan exposure. Two patients developed grade 1 acute GVHD of skin and two patients developed limited chronic GVHD. Three of these patients experienced full dose melphalan AUC between 3.5-6.5*ug/mL. The remaining one patient with limited chronic GVHD had full dose melphalan AUC of 9.5*ug/mL.

### (iv) Personized Full Dose Melphalan Adjustment

Interim analyses were completed after enrollment of 5 patients and 10 patients respectively to validate data following 25% and 50% of intended patient enrollment, and these interim analyses results provided the rationale for adjustment of full dose melphalan in patients at high risk of toxicity. In patients with impaired organ function at baseline, when the test dose PK predicted full dose AUC was >7.0 h*μg/mL, full dose of melphalan was adjusted to limit likelihood of toxicity. Six patients undergoing 7 transplants (one patient underwent HCT twice) received adjusted melphalan full dose and all had either impaired renal or liver function or both. Median test dose clearance was considerably lower than rest of the cohort (18.5 L/hr./70 kg, range 11.3-35.9 L/hr./70 kg) and median test dose AUC was higher than rest of the cohort (0.84 h*μg/mL, range 0.7-1.45 h*μg/mL). Correspondingly, test dose PK predicted AUC for standard full dose was considerably higher in these patients than rest of the cohort with a median AUC of 13.8 h*μg/mL (range, 11.1-16.4 h*μg/mL). Four of these patients had significantly impaired renal function with a GFR range of 24-43 ml/min/1.73 m²). One of the patients with renal impairment also had liver dysfunction (total bilirubin of 26 mg/dL, normal range 0.1-1.2 mg/dL) and underwent HCT twice with similar degree of organ impairment on both occasions. The remaining two patients had liver dysfunction; one patient had total bilirubin of 15.5 mg/dL and the other patient had sclerosing cholangitis of the liver. Patients with significantly impaired organ function were taken to transplant as a last-resort treatment option. Their organ dysfunction in part was secondary to their primary immune deficiency disorder and was presumed to improve post-transplant.

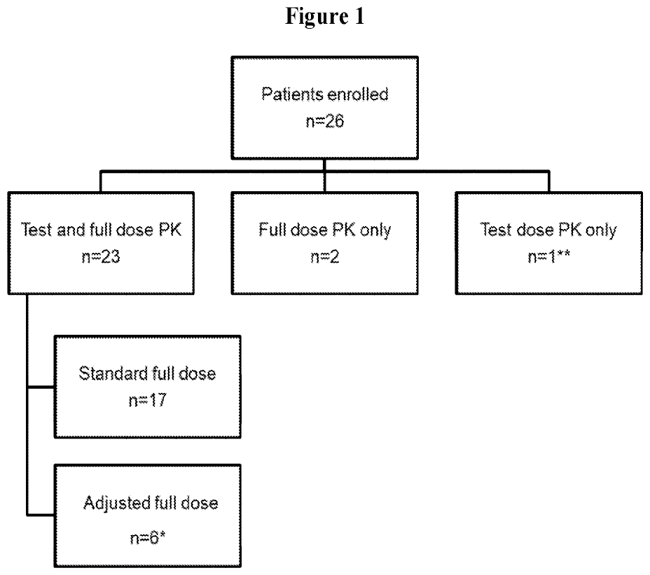

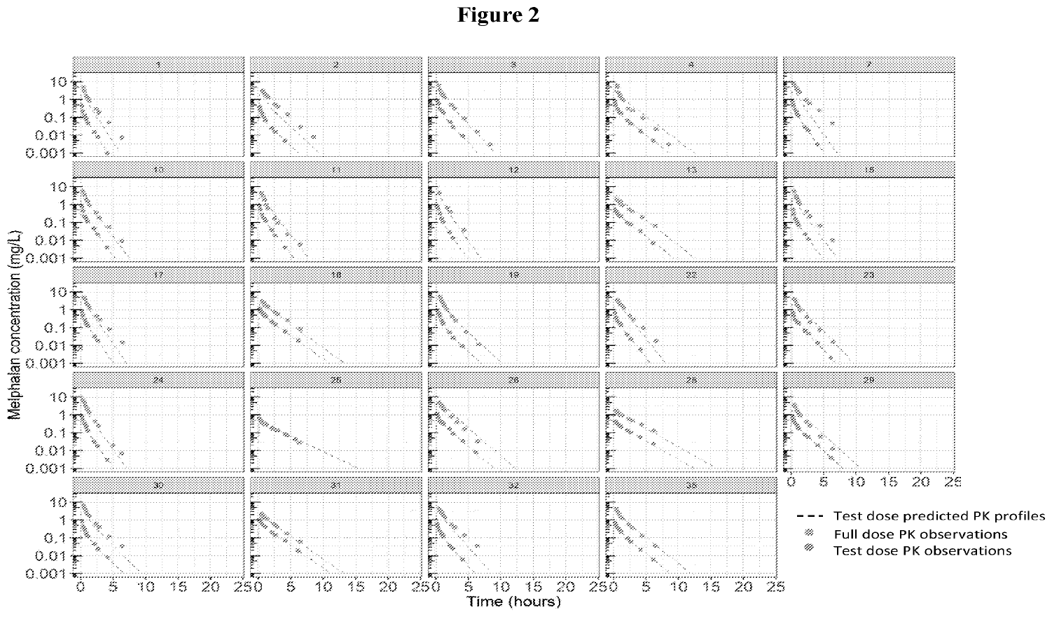

Five of these patients received adjusted full dose melphalan at 29% to 70% of standard dose (dose range, 32-99 mg/m²), with the desired target AUC between 4.0-5.5 h*μg/mL. Actual observed AUC with the adjusted dose was between 3.6-5.4 h*μg/mL, acceptably close to the desired range and also similar to the AUC achieved by majority of patients with normal organ function as shown in FIG. 1. All 5 patients engrafted with ≥99% donor chimerism and without excess toxicity. Grade 3 mucositis was observed in all 5 patients. None of the patients developed VOD. In the sixth patient who underwent HCT twice, full dose melphalan was 0.44 mg/kg (9.3% of standard dose) to achieved AUC of 1.1 h*μg/mL for his initial HCT. A lower target AUC was selected to minimize risk of VOD as the patient had severe liver impairment at baseline. The patient engrafted with 79% whole blood donor chimerism but subsequently developed secondary graft failure. Notably, the patient did not develop VOD of the liver or other excessive toxicity. The patient subsequently underwent a second HCT after 3 months with similar degree of renal and hepatic impairment and received full dose melphalan of 1.2 mg/kg (25.5% of standard dose) to achieve a target AUC of 4.5 h*μg/mL. Actual observed AUC was 3.9 h*μg/mL. The patient engrafted with 100% donor chimerism without developing VOD of the liver or other excessive toxicity.

### (v) Prediction Performance of Melphalan Test Dose by PK

Twenty one patients undergoing 22 transplants received both melphalan test dose and full dose. Of these, 15 patients had normal organ function and 6 patients underwent 7 transplants with significant impaired organ function at baseline. Melphalan test dose had robust prediction performance in patients with significant impaired organ function at baseline when full dose melphalan was adjusted based on baseline organ impairment. There was excellent correlation between predicted AUC and observed full dose AUC(R²=0.78) in patients receiving test dose PK guided adjusted full dose (FIG. 7A), with all (100%) patients achieving AUC in the desired target range. Test dose PK guided adjustment was able to significantly reduce melphalan exposure as shown in FIG. 7B. If these patients were given standard full dose, the full dose AUC would have been significantly higher at 11.0±3.6 h*μg/mL compared to the observed AUC of 3.9±1.4 h*μg/mL based on the test dose PK guided adjustment.

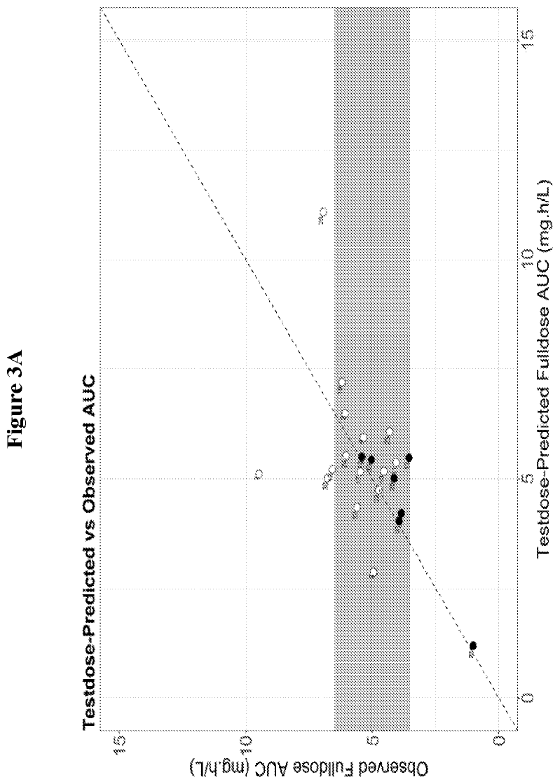

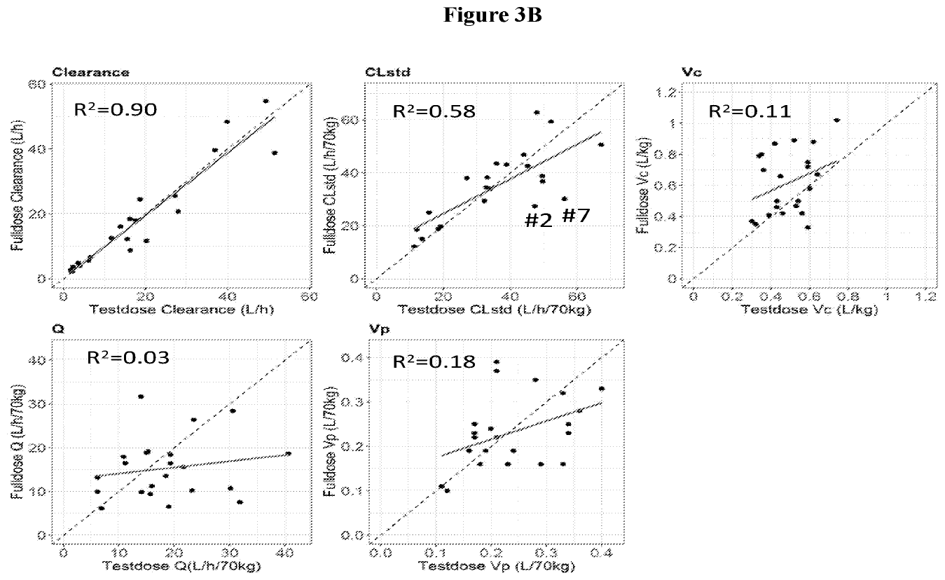

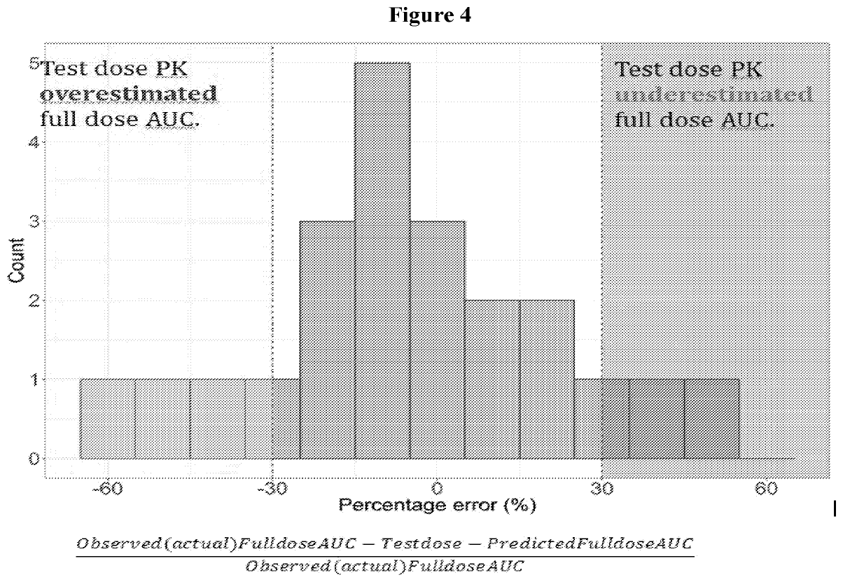

In the remaining 15 patients with normal organ function, melphalan test dose reliably predicted full dose AUC in 10/15 (66.7%) patients with a prediction error of less than 30%. Melphalan test dose PK either overestimated (predicted AUC more than observed AUC) or underestimated (predicted AUC less than observed AUC) the full dose AUC by >30% in the remaining 5 patients (33.3%) as shown in FIG. 4. Considerable variability between test dose and full dose melphalan clearance was also observed in these 6 patients. Correlation of test dose and full dose melphalan clearance is shown in FIG. 3B.

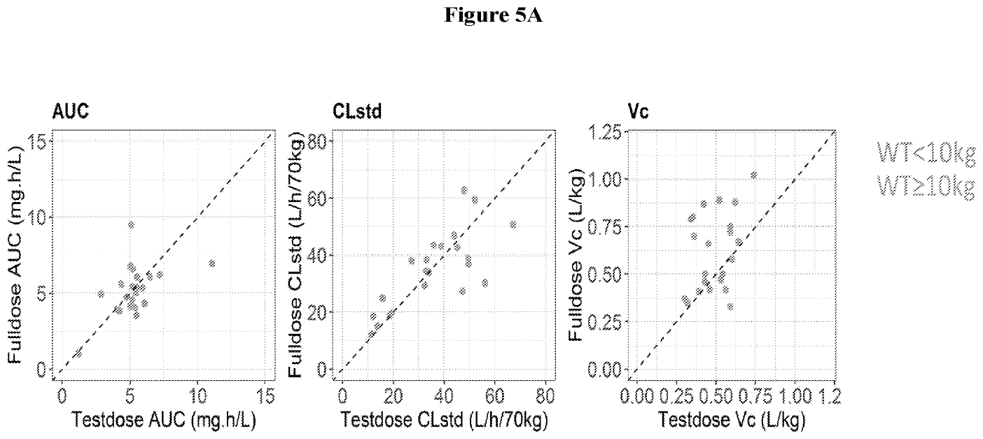

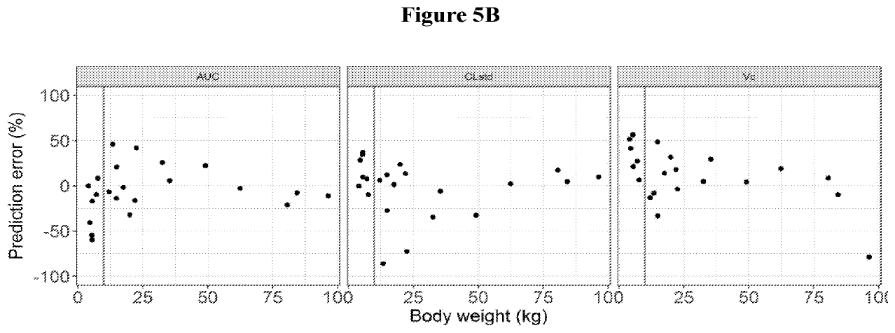

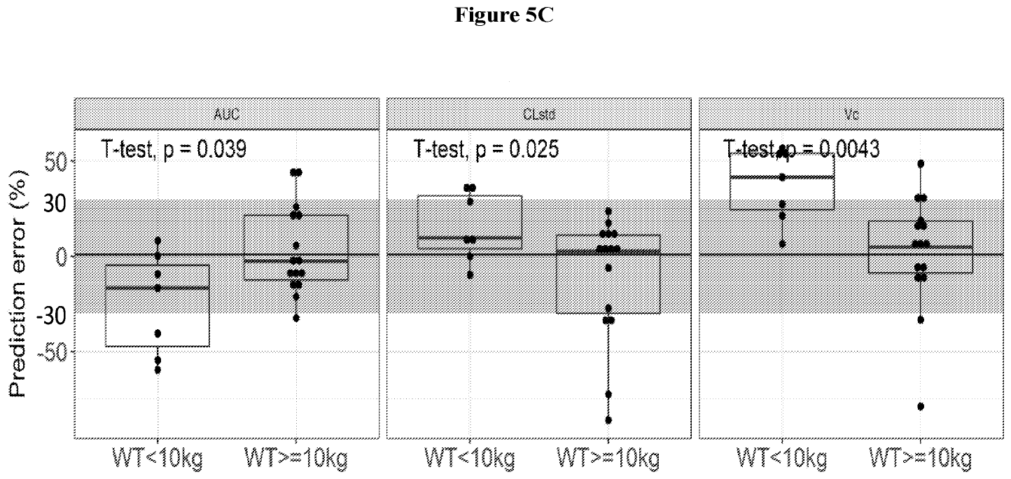

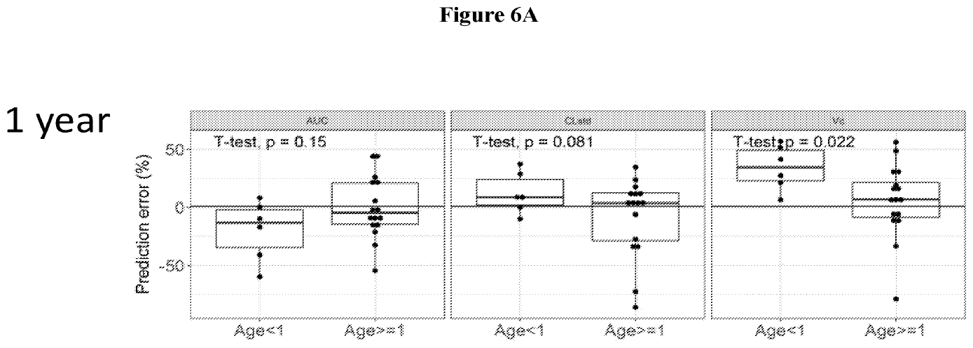

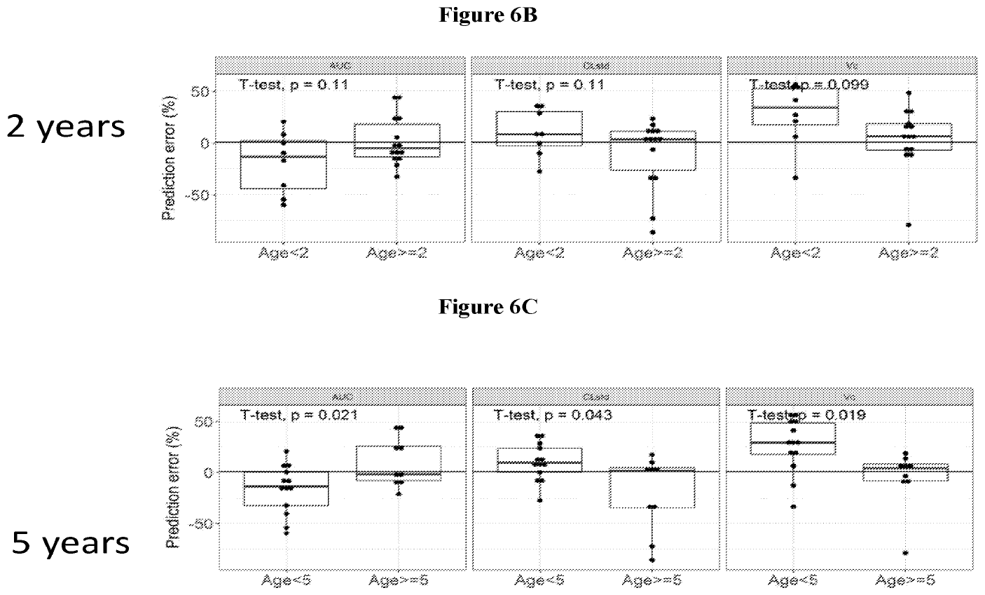

Further analysis revealed that body weight and age significantly affected prediction performance. In patients <10 kg or age <5 years, test dose PK significantly underestimated full dose clearance (T-test, p=0.025) and body weight adjusted volume of distribution (T-test, p=0.039), resulting in overestimation of full dose AUC (T-test, p=0.025) as shown in FIGS. 5A-C and FIGS. 6A-6C.

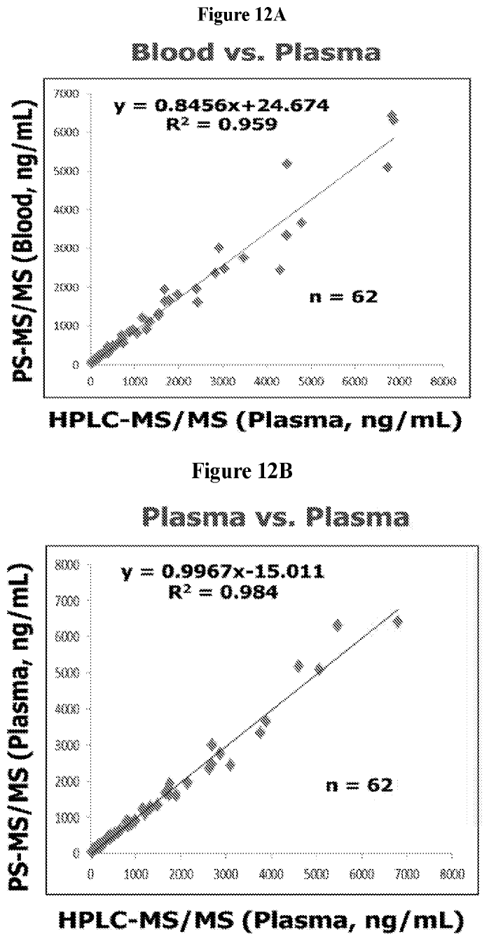

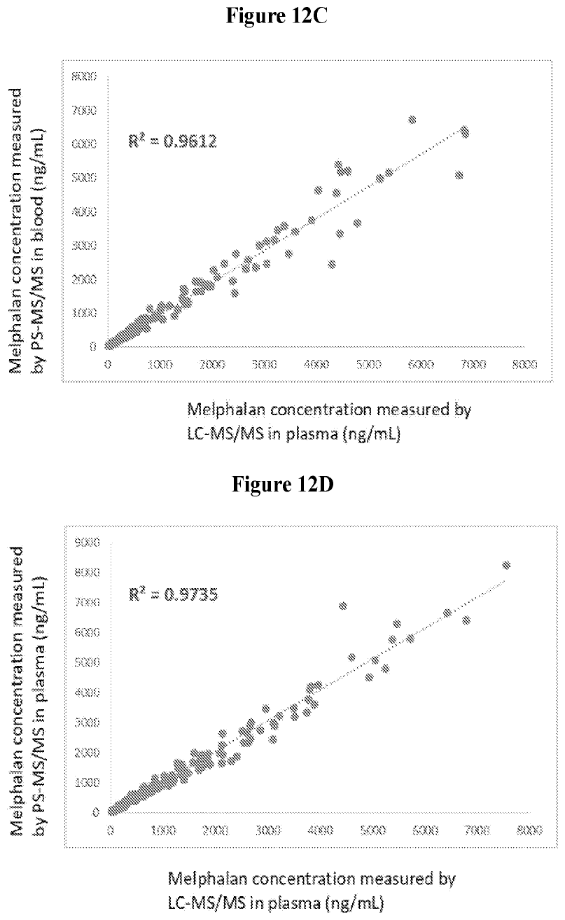

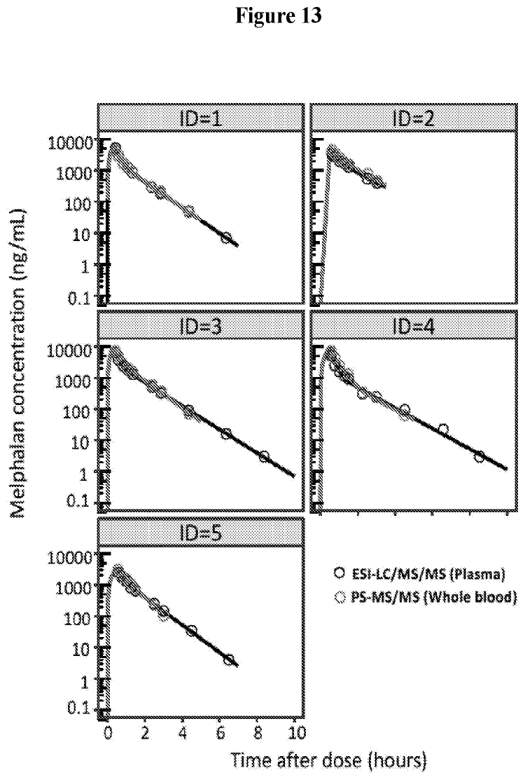

Melphalan concentrations obtained by PS-MS/MS and LC-ECI-MS/MS showed excellent correlation (FIGS. 12C-D). There was excellent correlation between blood melphalan concentrations measured by PS-MS/MS and plasma melphalan concentrations measured by conventional LC-MS/MS (n=192, R2=0.96) as shown in FIG. 12C. There was also excellent correlation between plasma melphalan concentrations measured by PS-MS/MS and plasma melphalan concentrations measured by conventional LC-MS/MS (n=208, R2=0.97) as shown in FIG. 12D.

### Discussion

Alemtuzumab, fludarabine and melphalan containing RIC regimen can be used in children undergoing allogeneic RIC HCT for non-malignant disorders. Melphalan is the predominant contributor of transplant related toxicity in this setting. This study describes melphalan PK in children and young adults undergoing HCT for non-malignant disorders using RIC with alemtuzumab, fludarabine and melphalan and to evaluate feasibility of a test dose PK guided precision dosing in this setting.

Busulfan experience in HCT setting has shown that test dose PK can reliably predict patients at increased risk of toxicity from higher systemic exposure, thereby allowing for full dose adjustment. In this study, predicted AUC for standard full dose melphalan was considerably higher in patients with significantly impaired renal or liver function. None of the patients in this study experienced full dose AUC >9.5 h*μg/mL to ascertain the full scope of toxicity. The present study avoids high exposure by adjusting the full dose of melphalan using results of test dose PK in all patients with baseline organ dysfunction. It is notable that adjusted full dose was 29% to 70% of standard dose demonstrating that significant dose reduction was needed to achieve desired AUC. Since the extent of organ impairment can vary, personalized dosing is required to achieve an AUC in the desired range and fixed dose reduction (e.g., 30% or 50%) may not be optimal in patients with impaired organ function. Moreover, higher melphalan exposure with fixed dose reduction would potentially increase risk of toxicity and lower than desired AUC may be insufficient to facilitate engraftment. In this study, test dose PK guided precision dosing allowed for transplant patients who may not have been eligible for transplant due to poor organ function. Overall, the results reported herein demonstrate the feasibility of precision dosing using test dose PK guided melphalan full dose adjustment in patients with impaired organ function undergoing HCT.

In patients with normal organ function undergoing HCT, test dose PK reliably predicted exposure from full dose of melphalan in two thirds of the patients. At an AUC range between 3.5 to 6.5 h*μg/mL, all patients achieved successful engraftment with full donor chimerism. Importantly, melphalan exposure at this AUC range was well tolerated without excess toxicity, with gastrointestinal mucositis being the most common side effect. This is an important consideration higher melphalan exposure would lead to a survival benefit in patients. Nath et al., Br J Clin Pharmacol. 2016; 82:149-159. In non-malignant disorders however, the role of melphalan is to create enough ‘marrow space’ to facilitate engraftment, unlike in malignant disorders where higher exposure is desirable in order to eradicate malignant cells. Higher melphalan exposure at the expense of increase in toxicity is therefore difficult to justify for non-malignant disorders. The present results suggest that AUC between 3.5-6.5 h*μg/mL is likely sufficient to facilitate engraftment with full donor chimerism and should be well tolerated without excess toxicity.

Interestingly, test dose PK was unable to reliably predict exposure from full dose of melphalan in one-third of the patients with normal organ function due to variation in clearance between test and full dose. In fact one of these patients developed grade 4 gastrointestinal mucositis following full dose of melphalan at an AUC of 9.5 h*μg/mL. This demonstrates a definite need for test dose PK-based precision dosing in patients with normal organ function also. Chemotherapeutic agents used between test dose and full dose melphalan during the preparative regimen have been reported to alter PK of the full dose. Nath et al. Br J Clin Pharmacol. 2005; 59:314-324; Gouyette et al. Cancer Chemother Pharmacol. 1986; 16:184-189, and Peters at al. Chemother Pharmacol. 1989; 23:377-383. Role of age and weight also needs to considered as prediction performance was sub-optimal in young children, i.e. children <10 kg and age <5 years. One way to improve this would be to use a larger test dose in younger patients. Changing the timing of test dose PK to after Campath® and fludarabine would also improve prediction performance. Similar to experience with Busulfan, the test dose melphalan PK guided approach disclosed herein is expected to be able to be utilized clinically in all patients.

Additionally, the present study also validated a novel real time PS-MS/MS assay, which has significant benefits over conventional methods, especially for the pediatric population. The small amount of blood required for measuring melphalan concentration would particularly benefit infants and very young children, where blood volume is often an obstacle for PK assessment. Furthermore, the rapid turnaround time for measuring melphalan would allow for real time monitoring and PK guided dose optimization in different transplant settings including malignant and non-malignant disorders.

In sum, this study reported melphalan pharmacokinetics (PK) analysis in children and young adults undergoing allogeneic HCT with a fludarabine, melphalan and alemtuzumab based RIC regimen for non-malignant disorders. Melphalan exposure between 3.5-6.5 h*μg/mL AUC was found to be well tolerated and likely sufficient to facilitate engraftment with full donor chimerism. Melphalan test dose PK can reliably predict full dose PK in patients with impaired organ function and allows for the accurate adjustment of full dose melphalan to avoid excess toxicity. Additionally, a novel and rapid paper spray MS assay has been validated for PK guided melphalan dose adjustment.

### Example 2: Real-time PK Study of Melphalan in Whole Blood by PS-MS/MS Method

Melphalan (4-[Bis(2-chloroethyl)amino]-L-phenylalanine, Alkeran®) is a bifunctional alkylating agent that inhibits DNA and RNA synthesis, cross-links strands of DNA and acts on both resting and rapidly dividing cells including tumor cells. High-dose melphalan is an important component of many hematopoietic stem cell transplantation (HSCT) preparative regimens to facilitate engraftment. Shaw P J, et al., Bone Marrow Transplant 1996; 16: 401-5 and Michel G, et al., Bone Marrow Transplant. 1988 March; 3(2):105-11. However, the toxicity of high dose melphalan is profound, and can be life threatening at times, affecting overall transplant outcomes. Although the pharmacokinetics (PK) of melphalan has been studied in animal models and in adult patients, such as Moreau P, et al., Br J Haematol 1996; 95: 527-30, Kuhne A, et al., Clin Pharmacol Ther 2008; 83: 749-757, and Sham P J, et al., Bone Marrow Transplantation. 2014, 49: 1457-1465, limited data exists in pediatric patients especially those undergoing allogeneic HSCT. Nath C E, et al., Br J Clin Pharmacol 2004; 59: 314-24. Personalized drug dosing would allow administration of the optimal chemotherapy dose to ensure engraftment of transplanted cells, while minimizing the toxicities.

Some analytical methods have been developed to quantify melphalan in biological samples. Typically, HPLC coupled with UV, fluorescence or electrochemical detection has been applied for pharmacokinetic assessment of melphalan so far, such as Bosanquet A G, et al., J of Chromatography, 232 (1982) 345-354, Pinguet F, et al., J of Chromatography B, 686 (1996) 43-49, Muckenschnabel I, et al., European Journal of Pharmaceutical Sciences, 5 (1997) 129-137, and Wu Z Y, et al., Journal of Chromatography B, 673 (1995) 267-279. LC-MS/MS assays for this drug, however, are using relatively complicated sample treatment or long chromatographic run time as indicated in Davies I D, et al., Chromatographia, 2000, 52, 51, 92-97, Mirkou A, et al., Journal of Chromatography B, 2009, 877, 3089-3096, and Sparidans R W, et al., Journal of Chromatography B, 2011, 879, 1851-1856. Most common problem encountered is that melphalan is unstable in blood and plasma at ambient temperature. It is hydrolyzed to the monohydroxymelphalan, which in its turn, is hydrolyzed to a stable compound, the dihydroxymelphalan. Nevertheless, these assays are not applied directly to human blood samples and also result in longer turnaround time, which may delay adjustment of dose that is critical, especially for pediatric patients.

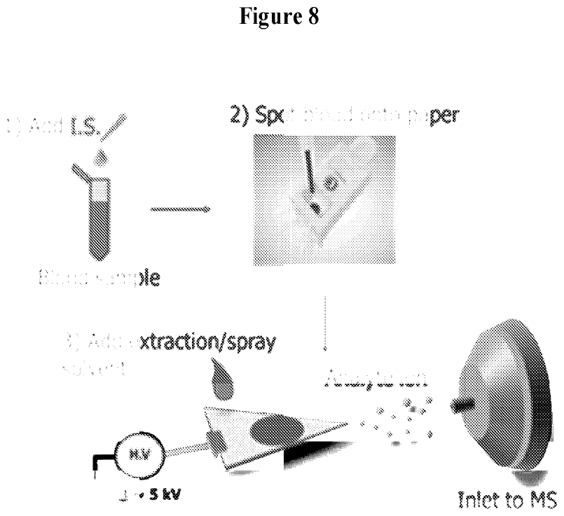

Paper spray (PS) is an ionization method that allows rapid quantitative analysis of pharmaceutical drugs by mass spectrometry directly from biological samples, including whole blood without the need for prior sample preparation or separation. See, e.g., Liu J J, et al., Anal Chem 2010; 82:2463-2471, and Wang H, et al., Angew Chem Int Ed 2010; 49:877-880. Sample extraction and ionization are all performed in automated fashion by the PS ion source from a paper substrate stored within a single-use cartridge. Briefly, paper spray-tandem mass spectrometry (PS-MS/MS) is performed by depositing whole blood and internal standard (I. S) mixture onto paper and allowing it to dry. An appropriate solvent is applied to the rear of the paper so that it flows through the dried blood spot (DBS) sample by capillary action. A high voltage (3-5 kV) is applied to the moist paper, inducing an electrospray at the sharp tip of the paper; the solvent evaporates from the droplets generating gas phase ions of the analyte molecules, which can then be detected by a mass spectrometry as shown in Liu J J, et al., Anal Chem 2010; 82:2463-2471. A schematic illustration of paper spray for MS analysis was shown in FIG. 8. The entire analysis time is only a few minutes which permits real-time analysis and rapid data reporting. Compared to the conventional LC-MS/MS methods, this new method has the additional advantages of significant reduction of solvent/reagent waste and elimination of carry-over.

Described herein are development and validation of a rapid PS-MS/MS method suitable for measuring the anti-cancer drug, melphalan, in small samples of blood, compared with the conventional LC-ESI-MS/MS method. The feasibility of this approach has been investigated for its application in clinical laboratory settings for real-time PK in order to select appropriate dosage regimen, for example, for pediatric patients who undergoing HSCT.

### Materials and Methods

### (i) Preparation of Reagents, Standards, and QCs

Melphalan powder (purity >95%) was from Sigma-Aldrich. Stable isotope labeled internal standard (SIL-IS) of [²H₈]-melphalan dihydrochloride (isotopic purity >98%) was from Toronto Research Chemicals. HPLC grade water, acetone, and ethanol were from Fisher Scientific. 2,2,2-Trifluoroethanol was from Sigma-Aldrich. Melphalan powder was carefully weighed and dissolved in methanol to obtain a 5 mg/mL stock solution and were stored at −80° C. The stock was further diluted in methanol to obtain working stock solutions. Calibration and quality control standard solutions of mephalan at various concentrations were prepared freshly and obtained by spiking pure melphalan solutions into drug-free whole blood and plasma respectively, keeping the organic solvent content at <5%.

### (ii) Patient Samples

Patients undergoing HSCT were administered by intravenous infusion a low dose of melphalan (referred to as the ‘test’ dose) to first determine each patient's individualized pharmacokinetics of the drug. After that the personalized optimal full dose was calculated from these measurements and then administered. The test dose equated to 10% of the expected standard full dose for each patient. Blood samples for pharmacokinetic measurement were collected after the test dose and again after the full standard dose of melphalan. Samples were collected at baseline, 5-10 mins before drug infusion, and then 5 min, 15 min, 30 min, 45 min, 1 h, 2h, 2.5h, 4h, and 6h post-infusion. The blood samples were collected on ice and immediately transferred to the lab for PS-MS/MS analysis after collection to minimize drug degradation. Plasma samples from the same blood draw were also obtained after centrifugation at 3000 rpm for 15 min and stored at −80° C. degree until analyzed by both PS-MS/MS and LC-ESI-MS/MS.

All samples were kept on ice during the sample preparation. Fresh drug free human citrate whole blood (380 μL) was used to prepare calibration standards at concentrations of 0, 25, 50, 100, 500, 1000, 5000, and 50000 ng/mL (0-163.9 μmol/L), and QC samples at concentrations of 50, 250, 2500, and 25000 ng/mL. The internal standard, [²H₈]melphalan (20 μL of a 20 μg/mL, or 65.5 μmol/L) was added and the sample mixed by vortexing. From these samples, 12 μL was spotted onto the disposable paper cartridges. (Prosolia Inc.). Drying of the blood spots was accelerated by placing the cartridge on a heated block (˜37° C.) and under a stream of nitrogen. Evaluation of the procedure was performed using triplicate blood spotted cartridges. The same procedure was used for plasma samples, where 10 L was spotted onto paper cartridges.

(iii) Paper Spray Ionization Tandem Mass Spectrometry (PS MS/MS) Analysis

PS-MS/MS was performed on an automated PS ion source (Prosolia, Inc. Indianapolis, Ind.) interfaced with a TSQ Quantum Ultra mass spectrometer (Thermo Scientific, San Jose, Calif.). This source serves the combined functions of an auto-sampler and ion source, in automatically loading the cartridges, delivering the solvent, positioning the cartridge in line with the MS inlet (4 mm from the inlet), and ejecting the spent cartridge after completion of the analysis. The ion source was programed to deliver the extraction/spray solvent comprising a mixture of ethanol/acetone/trifluoroethanol/H2O (40/20/20/20, by vol) to the cartridge at an optimized flow rate. A stepwise high voltage (2700-3000 V ramped over 1 min) was applied to the paper, inducing an electrospray at the triangular paper tip; the solvent evaporates from the droplets generating gas phase ions of the analyte. Espy R D et al., Analyst 2012; 137:2344-2349, Manicke N E, et al., J Am Soc Mass Spectrom 2011; 22:1501-1507, and Yang Q, et al., Int. J Mass Spectrom 2012; 312:201-207. The MS conditions for MRM were first optimized using continuous infusion of melphalan and [²H₈]melphalan solutions into the ESI source using a syringe pump, and MRMs selected accordingly. The average time required per sample analysis was 3 mins, which included extraction and data collection. A total of 50 scans in positive ion mode were acquired over 1 min for each m/z transition monitored. The transitions m/z 305.3 →246.2 for melphalan and the corresponding transition m/z 313.3→254.2 for the I.S. were used for quantification, while the transition m/z 305.3→194.2 served as a qualifier for melphalan confirmation. Specificity was established when the quantifier and qualifier SRM ions were averaged over the entire scan time and ratio of the two were within ±25%. XCalibur software was used to control the instrument and process data. A calibration curve was generated by plotting the ratio of the area under the curve (AUC) of melphalan to IS against the concentrations of melphalan by weighted (1/x) least-squares linear regression, and this was used for calculation of all patient sample concentrations and QC samples.

(iv) Data Collection and Analysis

Data was collected for 60s by ramping the spray voltage from 2700V to 3000V, resulting in a total of 50 scans for each SRM channel. The average time required per sample analysis was 3 minutes, which included extraction and data collection. The major fragmentation of the 2 SRMs, m/z 305.3→246.2 for melphalan and m/z 313.3→254.2 for the I.S. was used for quantitation, while the other transition was used for confirmation (Table 6). A calibration curve was generated by plotting the area under the curve (AUC) ratios of melphalan to IS versus the actual concentrations of melphalan by weighted (1/x) least-squares linear regression, and was used for calculation of all patient sample concentrations as well as QCs. Results were confirmed when the quantifier and qualifier SRM ions were averaged over the entire scan time and ratio of the two was within mean±25%.

TABLE 6
TSQ Ultra SRM Parameters

| | Parent | Product | | | | | | Tube |
|---|---|---|---|---|---|---|---|---|
| Name | ion | ion | Width | Time(s) | CE | Q1 | Q3 | lens |
| Melphalan | 305.3 | 246.2 | 0.01 | 0.1 | 23 | 0.4 | 0.7 | 126 |
| | 305.3 | 194.2 | 0.01 | 0.1 | 32 | 0.4 | 0.7 | 126 |
| [²H₈]— | 313.3 | 254.2 | 0.01 | 0.1 | 23 | 0.4 | 0.7 | 130 |

(v) Pharmacokinetic Analysis

Each patient's pharmacokinetic profile was determined using WinNonlin v6.4 (Pharsight, Mountain View, Calif.). The area under the curve (AUC) of the blood concentration-time profile was estimated by linear trapezoidal integration using standard equations. Graphical individual PK evaluation was performed using R v3.0.3 and MW\Pharm 3.82 (MEDIWARE a.s., Prague, Czech Republic).

(vi) Method Validation

The PS-MS\MS method was validated in accordance with the FDA guidance for Bioanalytical Method Validation (fda.gov/downloads/Drugs/Guidance/ucm070107.pdf, May 2011), and Bansa S, et al., AAPS J., 2007, 9, 109-114. Specifically, three analytical runs were processed and analyzed to assess sensitivity, reproducibility, accuracy and precision. Each analytical run contained 7 calibration points defining the analytical range (n=2 at each level), two control blanks (blanks without IS), two zero standards (blanks with IS only), and quality control samples (n=6 at each level). The pre-defined acceptance criteria for a successful analytical run followed standard guidelines. The assay accuracy was evaluated by comparing results obtained by PS-MS/MS to those obtained by a validated in-house LC-MS/MS melphalan assay for the same samples, wherein melphalan samples were measured by liquid chromatography-tandem mass spectrometry (LC-MS/MS) with selected-reaction monitoring and with the use of stable isotopic-labeled [2H8]-melphalan as the internal standard. Samples were analyzed with the LC20AD HPLC system (Shimadzu) coupled to the TSQ Quantum Ultra Triple Quadrupole Mass Spectrometer (Thermo Scientific). Chromatographic separation was achieved on a 150×2 mm Prodigy 5 μm ODS-2 150Δ LC column (Phenomenex). A gradient mobile phase was used with a binary solvent system, which changed from 25% mobile phase B (acetonitrile/0.1% formic acid/) to 5% mobile phase A (water/0.1% formic acid) at a flow rate of 0.6 ml/min. The total run time was 10 min, and the injection volume was 10 μL. The optimal signal for the analytes was achieved in positive ion mode with the following instrument settings: spray voltage: 4 kV; sheath gas pressure: 35; auxiliary gas flow: 10; and capillary temperature: 350° C. Argon was used as the collision gas. Data were acquired and processed with Xcalibur 2.2 (Thermo Scientific). The lower limit of quantification (LOQ) was 2 ng/mL and the calibration curve was linear over the concentration range of 2-1000 ng/mL for melphalan.

### Results

(i) Optimization

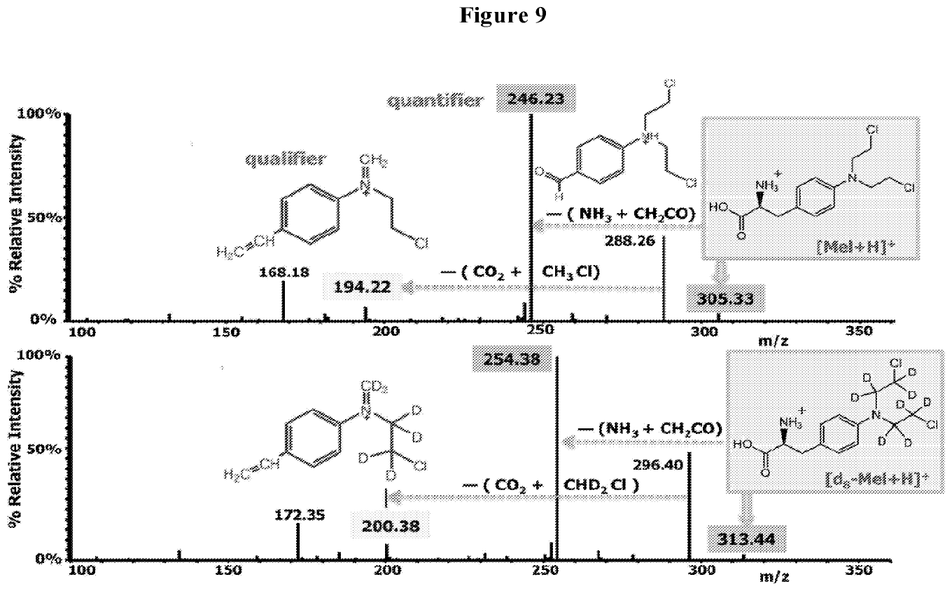

Melphalan gives an abundant protonated molecular ion of m/z 305.2 and following collisional activation, a predominate product ion of m/z 246.2 corresponding to the loss of NH₃+CH₂CO) which was chosen as the quantifier. A less abundance but specific product ion of m/z 194.2 corresponding to the loss of [CO₂+CH₃Cl] was used as the qualifier transition. The fragmentation pathways were further confirmed by the mass shift for internal standard, [²H₈]-melphalan, CID spectra of the protonated melphalan and [²H₈]-melphalan as shown in FIG. 9. An extraction/spray solvent composition comprising ethanol/acetone/trifluoroethanol/water (40/20/20/20, by vol) was found to be optimal to move and ionize melphalan and its internal standard from the dried blood spot with maximum intensity and duration. Continuous syringe pump infusion was used to determine the optimal ion source and MS instrument settings for SRM analysis (Table 6). Cartridges spotted with pure melphalan and [²H₈]-melphalan solution were dried and used to confirm the settings.

(ii) Chronogram and Interference

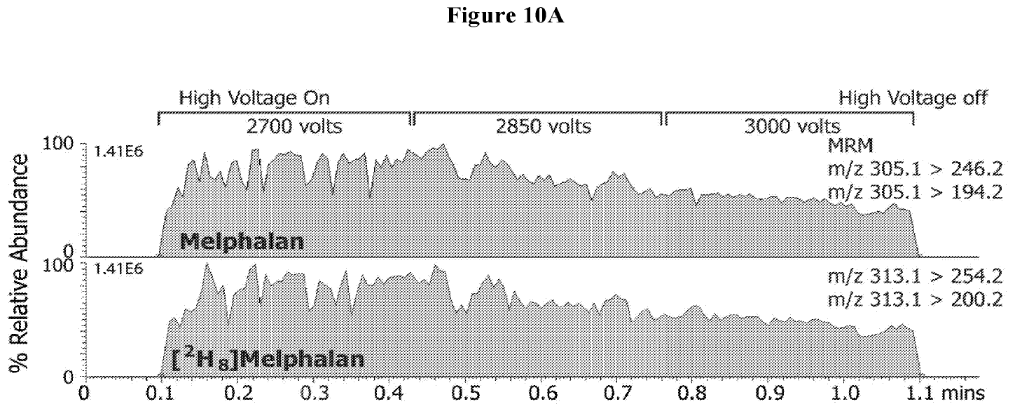

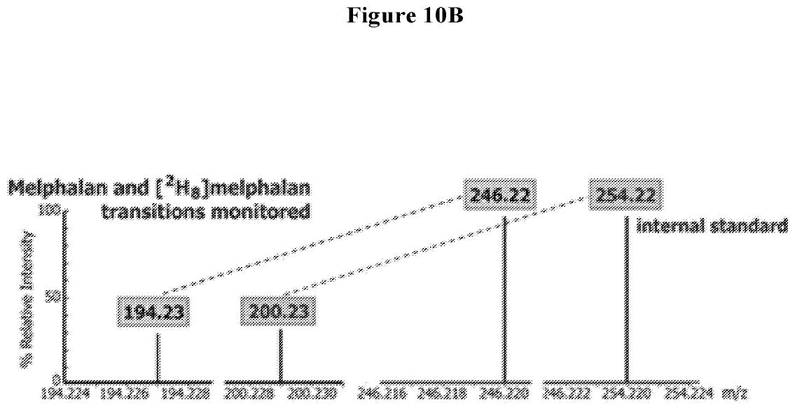

A representative PS-MS/MS SRM chronogram from a patient sample containing melphalan is shown in FIG. 10. During paper spray analysis, the compounds extracted from the DBS are introduced directly to the mass spectrometer with essentially no prior sample preparation. Because of the lack of any sample pretreatment, the effect of interferences arising from products of hemolysis, lipids, and other blood components on the melphalan and IS ion intensities was evaluated and compared with the response obtained from the pure compounds in methanol. There was no difference in the AUC or the shape of the chronogram based on the presence of absence of a blood matrix, which concurs with the findings reported by Shi et al for several immunosuppressants (Shi R Z, Clinica Chimica Acta 2015; 441: 99-104 and Clin Chem 2016; 62(1): 295-9). Some variability in the shape of the chronogram was observed but this was not consistently associated with any particular type of sample and was compensated for by the stable-labeled IS that was used for quantification. Our experience from the analysis of >200 pediatric patient blood samples showed that no failures were accounted for by inadequate ion response and/or failure of the ion ratio criterion for positive identification of melphalan. Interestingly, Shi et al (Clinica Chimica Acta 2015; 441: 99-104) reported up to 10% of samples required reanalysis due to the failure of the sample cartridges, something we have not experienced.

(iii) Linearity and Lower Limit of Quantification

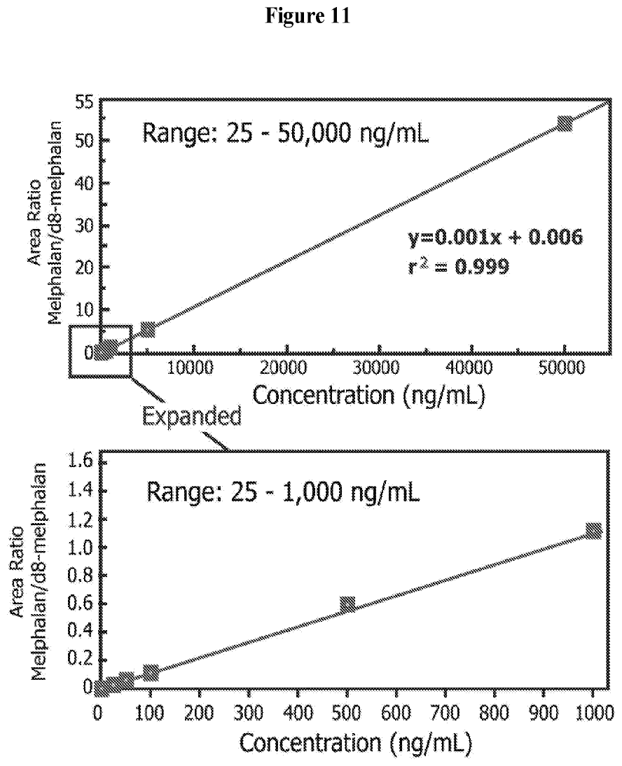

The calibration curves for melphalan quantification were obtained by plotting the area under curve (AUC) for the 305.3→246.2: 313.3→254.2 ion pairs vs the concentration of melphalan. Calibration curves were analyzed by weighted least-squares linear regression analysis and were linear over the range of 25-50,000 ng/mL (see FIG. 11). The slopes, intercepts, and coefficient of determination (r²) from the validation are summarized in Table 7. The lower limit of quantitation was defined to be the lowest analyte concentration that gave a signal 10 fold greater than drug-free blank blood, had a relative standard deviation (RSD) of <20%, and was within 20% of the expected value. The LLOQ was then conservatively set at 50 ng/mL, indicating adequate sensitivity to quantify the melphalan concentrations in the therapeutic range and over expected blood appearance/disappearance concentrations in a PK study.

(iv) Accuracy and Precision

The inter-run precision and accuracy for calibration curves from the three analytical runs are listed in Table 8. The inter-run accuracy expressed as % bias ranged from −1.2% to 7.3% (n=6). The inter-run reproducibility (% CV) ranged from 0.7 to 11.0% (n=6). For QC samples, the inter-run precision and accuracy from the three analytical runs are summarized in Table 8. The inter-run accuracy (% bias) ranged from 0.8% at the LLOQ to 5.7% at the QC HIGH (n=18). The inter-run precision (% CV) ranged from 7.9% at the LLOQ to 3.0% at the QC HIGH (N=18).

(v) Comparison of PS-MS/MS with ESI-LC-MS/MS

A cross-validation of the assay was assessed by comparing the melphalan concentrations in samples from patients administered the drug determined by PS-MS/MS with an in-house electrospray ionization LC-MS/MS method (see FIGS. 12A-12D). PS-MS/MS blood melphalan concentrations from 62 patient samples showed an excellent correlation (R2=0.959, n=62) with conventional LC-MS/MS methods (FIG. 12A), and PS measurement of plasma gave similar results with a correlation of R2=0.984 (FIG. 12B). These results indicate that this rapid PS-MS/MS method, which involves first drying the blood samples followed by a direct extraction into the instrument using a polar organic solvent yielded consistent and ‘accurate’ drug concentrations in blood. Similar comparisons have been previously reported for propranolol and atenolol and were explained by the likelihood that the plasma proteins are denatured during the drying process and any noncovalent interactions further disrupted by the addition of organic solvents, Manicke N E, et al., J Am Soc Mass Spectrom 2011; 22: 1501-7.

(vi) Recovery and Matrix Effect

Previous studies with PS-MS (Shi, R Z, et al, Clinica Chimica Acta 2015; 441: 99-104 and Clin Chem 2016; 62(1): 295-9, and Manicke N E, Int J. Mass Spectrom 2011; 300:123-9) have indicated that for some compounds the amount of analyte that is extracted and sprayed from the paper is relatively low. The low ion transmission is what limits the sensitivity of PS in general but for many therapeutic drugs PS-MS/MS has adequate sensitivity in the therapeutic range. In this study, recovery and matrix effects were evaluated by comparing the absolute signal responses of the IS, calculated as the AUC for the quantifier ion prepared in methanol versus whole blood. Calibration standards in methanol and drug free whole blood were prepared and analyzed on three separate days and were low but comparable to other analytes assayed using paperspray (Shi, R Z, et al, Clinica Chimica Acta 2015; 441: 99-104 and Clin Chem 2016; 62(1): 295-9). The results were highly reproducible and the stable isotope-labeled internal standard compensates for both incomplete recovery from the paper and background noise. For accurate quantification of melphalan in whole blood, calibrators were therefore prepared in matrix-matched whole blood and spotted to the paper spray cartridge immediately prior to analysis of patient samples. Differences in the matrix effect among different patient samples were also evaluated. Twenty blood samples from five patients were analyzed on different days. The average absolute signal from the internal standard was compared to the average signal obtained for all of the samples. None of the patient samples were significantly different from the pooled mean at the 95% confidence level.

TABLE 7
Back-Calculated Melphalan Concentration in Blood.

| Concentration | STD1 | STD2 | STD3 | STD4 | STD5 | STD6 | STD7 | Slope | Intercept | |
|---|---|---|---|---|---|---|---|---|---|---|
| (ng/mL) | 25 | 50 | 100 | 500 | 1000 | 5000 | 50,000 | (m) | (b) | r² |
| Run 1 | 27.4 | 54.3 | 101.9 | 518.8 | 1034.3 | 4874.6 | 50033.5 | 0.001 | 0.006 | 0.9998 |
| Run 2 | 28.4 | 50.7 | 99.1 | 506.7 | 1125.8 | 5362.3 | 49479.7 | 0.001 | 0.005 | 0.9991 |
| Run 3 | 22.8 | 49.4 | 108.5 | 502.5 | 1059.0 | 4962.2 | 49977.7 | 0.001 | 0.010 | 0.9996 |
| Run 4 | 26.1 | 50.7 | 99.1 | 489.9 | 976.3 | 4474.1 | 50568.7 | 0.001 | 0.005 | 0.9989 |
| Run 5 | 22.1 | 54.0 | 97.4 | 505.5 | 1127.5 | 5128.5 | 49731.0 | 0.001 | 0.008 | 0.9996 |
| Run 6 | 22.5 | 48.8 | 101.5 | 513.0 | 1116.2 | 4836.9 | 50017.1 | 0.001 | 0.010 | 0.9996 |
| n | 6 | 6 | 6 | 6 | 6 | 6 | 6 | | | |
| mean | 24.9 | 51.3 | 101.2 | 506.1 | 1073.2 | 4939.8 | 49968.0 | | | |
| S.D | 2.75 | 2.32 | 3.93 | 9.85 | 61.10 | 298.8 | 363.9 | | | |
| % CV | 11.07 | 4.52 | 3.88 | 1.95 | 5.69 | 6.05 | 0.73 | | | |
| % bias | −0.47 | 2.63 | 1.25 | 1.21 | 7.32 | −1.20 | −0.06 | | | |

Data was fit using a linear regression (y = mx + b) with a weighting of 1/concentration.

TABLE 8
Accuracy and Precision Data for
Melphalan Quality Control Samples

| Concentration | LLOQ | QOL | QMED | QHIGH |
|---|---|---|---|---|
| (ng/mL) | 50 | 250 | 2500 | 25,000 |
| Run 1 | 51.2 | 243.7 | 2649.1 | 26944.5 |
| | 50.8 | 247.2 | 2641.2 | 27123.8 |
| | 50.9 | 242.1 | 2620.0 | 26957.5 |
| | 54.7 | 248.7 | 2881.9 | 26428.9 |
| | 46.2 | 260.7 | 2855.8 | 26788.0 |
| | 48.2 | 253.7 | 2896.7 | 26512.8 |
| Run 2 | 44.6 | 239.9 | 2560.0 | 25765.4 |
| | 57.2 | 245.9 | 2493.8 | 26839.8 |
| | 43.9 | 255.5 | 2672.3 | 26367.7 |
| | 56.7 | 230.4 | 2394.6 | 26230.0 |
| | 57.4 | 228.9 | 2555.9 | 25105.7 |
| | 50.7 | 223.8 | 2656.4 | 24704.0 |
| Run 3 | 49.0 | 265.5 | 2687.5 | 27976.8 |
| | 50.2 | 239.6 | 2646.2 | 26967.5 |
| | 47.2 | 225.4 | 2519.3 | 25805.8 |
| | 49.0 | 234.6 | 2575.6 | 25746.1 |
| | 48.9 | 243.5 | 2606.8 | 26438.0 |
| | 50.5 | 246.7 | 2484.1 | 27056.4 |
| n | 18 | 18 | 18 | 18 |
| Mean | 50.4 | 243.1 | 2633.2 | 26431.0 |
| S.D | 4.0 | 11.6 | 135.8 | 785.0 |
| % CV | 7.90 | 4.78 | 5.16 | 2.97 |
| % bias | 0.81 | −2.76 | 5.33 | 5.72 |

LLOQ: low limit of quantitation;
QOL: quality control of low concentration;
QMED: quality control of medium concentration;
QHIGH: quality control of high concentration

(vii) Application in Clinical Real-Time Pharmacokinetic Study

This PS-MS/MS methodology has been successfully used to support a ‘real-time’ pharmacokinetic study of melphalan in children undergoing HSCT. Dosing decisions on melphalan for pediatric patients is largely empirical and based on scaling down from recommendations for adult dosing. Knowledge of the exact PK behavior of melphalan in pediatric patients would permit patient specific dosing decisions to minimize toxicity and improve efficacy of transplant outcomes. In order to minimize potential toxicity, which can be fatal in some patients, a strategy of first dosing the patient with a very low dose of melphalan designed to avoid toxicity was adopted and the PKs immediately determined. These data were then used to estimate the optimal therapeutic dose for each patient to avoid toxicity. This approach requires a very rapid analysis of blood samples collected over a reduced time period and immediate reporting of results to then compute the PK data and dose adjust at the bedside. In this regard PS-MS/MS with rapid analysis and minimal sample preparation has proven in our hands to be an ideal tool to facilitate this decision process as its simplicity and speed offers advantages over other conventional mass spectrometry approaches.

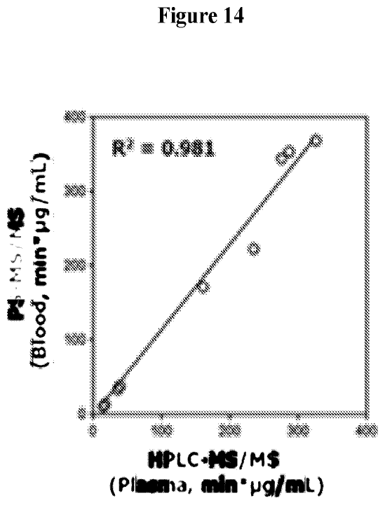

Blood melphalan concentrations determined by PS-MS/MS were used to compute melphalan PK in 5 young pediatric patients (age range 1.5-16.8 years) administered a test dose (10% of estimated full dose) and these data were compared with levels measured in the same patients administered the calculated full dose (Table 9). Excellent comparisons were obtained. We further visually compared the whole blood melphalan PK, for 5 patients after administration of the standard dose with plasma levels measured by LC-MS/MS and almost identical PK profiles were observed for each patient (FIG. 14). The only advantage of LC-MS/MS was the ability to detect melphalan at lower concentrations than was possible by PS-MS/MS. From these data, melphalan exposure expressed as AUC (h μg/mL) was calculated and the results summarized in Table 9. The calculated mean AUC in patients given the full dose was 4.9±1.5 h μg/mL by PS-MS/MS method and was 4.3±1.0 h*μg/mL by ESI-LC-MS/MS method (n=5), while the calculated mean AUCs in patients given the lower test doses were 0.41±0.23 h*μg/mL and 0.43±0.21 h μg/mL respectively (n=4). There was a linear correlation (R²=0.981) between the AUC determined by PS-MS/MS for whole blood and the LC-MS/MS concentrations for plasma.

In summary, a validated, simple, accurate and rapid PS-MS/MS method for measuring melphalan in whole blood is described. This method is applicable for measuring melphalan in pediatric patients where blood volumes, especially when repeat sampling is performed for PK studies, are limited. This PS-MS/MS assay involves minimal sample pretreatment, eliminates chromatography, and consequently results can be obtained in minutes once the whole blood is spotted on the paper cartridge. The incorporation of a stable-isotopically labeled internal standard accounts for variations in extraction recovery and matrix effects from the paper and the assay has high precision, accuracy and sensitivity in the therapeutic range for melphalan. We have successfully applied this approach over the last 4 years to support the real-time pharmacokinetic study of melphalan in children undergoing HSCT. To our knowledge, this is the first report to evaluate the clinical application of PS-MS/MS for ‘real-time’ AUC measurement and pharmacokinetically-guided individualized precision dosing strategy to improve overall HSCT outcomes for these patients.

TABLE 9
Bioavailability of Melphalan Calculated from the Area under the Curve (AUC)
in Patients Undergoing HSCT Measured during a Test Dose and Therapeutic Full Dose
Determined from Whole Blood Using PS-MS/MS Compared with Plasma by ESI-LC-
MS/MS

| | | | PS- | | | PS- | |
|---|---|---|---|---|---|---|---|
| | | | MS/MS | ESI-LC- | | MS/MS | ESI-LC- |
| | | | Whole | MS/MS | | Whole | MS/MS |
| | | Test Dose | blood | Plasma | Full Dose | blood | Plasma |
| | Age | mg | AUC | AUC | mg | AUC | AUC |
| Patient | (yr) | (mg/kg bw) | (h*μg/mL) | (h*μg/mL) | (mg/kg bw) | (h*μg/mL) | (h*μg/mL) |
| 1 | 12.6 | 19 mg | 0.18 | 0.22 | 190 mg | 3.7 | 3.9 |
| | | (0.43 | | | (4.32 mg/kg | | |
| | | mg/kg bw) | | | bw) | | |
| 2 | 5.6 | 5.81 (0.24 | 0.23 | 0.27 | 58 (2.9 | 5.7 | 4.6 |
| | | mg/kg bw) | | | mg/kg bw) | | |
| 3 | 2.6 | 7.6 (0.61 | 0.64 | 0.64 | 76 (6.08 | 6.1 | 5.4 |
| | | mg/kg bw) | | | mg/kg bw) | | |
| 4 | 16.8 | 29.3 (0.33 | 0.57 | 0.57 | 293 (3.29 | 5.9 | 4.8 |
| | | mg/kg bw) | | | mg/kg bw) | | |
| 5* | 1.5 | — | — | — | 44 (4.49 | 2.9 | 2.7 |
| | | | | | mg/kg bw) | | |
| Mean | | | 0.41 | 0.43 | | 4.9 | 4.3 |
| ±SD | | | 0.23 | 0.21 | | 1.5 | 1.0 |

*Patient 5 was administrated only the full therapeutic dose.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for determining a personalized full dose of a melphalan compound in a reduced intensity conditioning regimen (RIC) for a subject in need thereof, the method comprising:
(i) administering to the subject in need thereof a test dose of the melphalan compound, wherein the test dose of the melphalan compound is about 10% to about 30% of a standard full dose of the melphalan compound for use in RIC;
(ii) collecting blood samples before administration of the test dose of the melphalan compound and at multiple time points after administration of the test dose of the melphalan compound;
(iii) measuring the levels of the melphalan compound or a metabolite thereof in the blood samples;
(iv) calculating pharmacokinetic features of the melphalan compound based on the levels of melphalan or the metabolite thereof measured in step (iii); and
(v) determining a personalized full dose of the melphalan compound in the RIC for the subject based on the pharmacokinetic features calculated in step (iv).

2. The method of claim 1, wherein the melphalan compound is melphalan.

3. The method of claim 1, wherein the subject is in need of hematopoietic cell transplantation.

4. The method of claim 1, wherein the pharmacokinetic features of the melphalan compound comprises area under the curve (AUC), median clearance (CL), or both.

5. The method of claim 1, wherein the method further comprises (vi) subjecting the subject to a RIC comprising melphalan, and wherein the subject is administered with the melphalan at the personalized full dose determined in step (v).

6. The method of claim 5, wherein the RIC further comprises alemtuzumab and fludarabine.

7. The method of claim 1, wherein the method further comprises subjecting the subject to hematopoietic cell transplantation after step (vi).

8. The method of claim 1, wherein the subject is a human patient having a non-malignant disorder.

9. The method of claim 1, wherein the human patient has a hematologic disease.

10. The method of claim 9, wherein the hematologic disease is selected from the group consisting of an immune deficiency disorder, a hemoglobinopathy, bone marrow failure, a genetic metabolic disorder, and anemia.

11. The method of claim 10, wherein the bone marrow failure is a congenital bone marrow failure disorder or an acquired bone marrow failure disorder.

12. The method of claim 10, wherein the hemoglobinopathy is sickle cell disease.

13. The method of claim 1, wherein the subject is a human patient having hemophagocytic lymphohistiocytosis, severe combined immune deficiency, combined immune deficiency, aplastic anemia and/or bone marrow failure, sickle cell disease, immunodysregulation polyendocrinopathy enteropathy X-linked (IPEX) or IPEX-like syndrome, or erythropoietic protoporphyria.

14. The method of claim 1, wherein the subject is a human child.

15. The method of claim 14, wherein the human child is younger than 5 years.

16. The method of claim 14, wherein the subject is a human infant.

17. The method of claim 14, wherein the subject has a body weight lower than 10 kg.

18. The method of claim 14, wherein the test is up to about 30% of the standard full dose of the melphalan compound.

19. The method of claim 1, wherein the subject is a human adult.

20. The method of claim 1, wherein the subject is a human patient having an organ dysfunction.

21. The method of claim 20, wherein the human patient has liver dysfunction, kidney dysfunction, severe colitis, respiratory failure, cardiac dysfunction, or a combination thereof.

22. The method of claim 1, wherein the test dose is about 10% of the standard full dose of the melphalan compound.

23. The method of claim 1, wherein the blood samples are collected before administration of the melphalan compound and at multiple time points selected from the group consisting of about 5 minutes, about 15 minutes, about 30 minutes, about 45 minutes, about 60 minutes, about 2 hours, about 2.5 hours, about 4 hours, and about 6 hours after administration of the melphalan compound.

24. The method of claim 1, wherein the blood samples are collected at about 0.08 hour, 0.5±0.1 hour, 1.5±0.3 hours, and 4.0 hours after the administration of the melphalan compound.

25. The method of claim 1, wherein the blood samples are collected between 0.08-0.19 hour, 0.33-0.90 hour, 1.3-2.7 hours, and 3.6-4.0 hours after the administration of the melphalan compound.

26. The method of claim 1, wherein the levels of the melphalan compound or the metabolite thereof is determined by LC-MS/MS or paper spray (PS)-MS/MS.

27. The method of claim 4, wherein the median clearance is median body weight normalized clearance (CL_{STD}).

28. The method of claim 1, wherein the personalized full dose of melphalan determined in step (v) is based further on one or more of characteristics of the subject.

29. The method of claim 28, wherein the one or more characteristics of the subject comprise age, weight, disease condition, organ function, blood cell count, bone marrow cellularity, infectious status, congenital anomaly, clinical status, or a combination thereof.

30. The method of claim 29, wherein the organ function comprises liver function, kidney function, digestive tract function, lung function, cardiac function, or a combination thereof.

31. The method of claim 4, wherein the AUC is calculated by the trapezoidal method.

32. The method of claim 1, wherein the personalized full dose determined in step (v) result in a target AUC of about 3.5-6.5 h*μg/mL in the subject.

33. The method of claim 6, wherein in step (ii), at least a portion of the blood samples are corrected after administration of the alemtuzumab and/or fludarabine.
